(19) United States
(12) Patent Application Publication
(10) Pub. No.: US 2026/0130947 A1
(43) Pub. Date: May 14, 2026

(54) EXTRACELLULAR VESICLES FOR TREATMENT OF AMYOTROPHIC LATERAL SCLEROSIS

(71) Applicant: Aruna Bio, Inc., Athens, GA (US)

(72) Inventors: Steven L. Stice, Athens, GA (US); Emily Baker, Athens, GA (US); Amruta Narayanappa, Watkinsville, GA (US)

(73) Assignee: Aruna Bio, Inc., Athens, GA (US)

(21) Appl. No.: 19/442,667

(22) Filed: Jan. 7, 2026

Related U.S. Application Data
(63) Continuation of application No. PCT/IB2024/056590, filed on Jul. 5, 2024.
(60) Provisional application No. 63/512,483, filed on Jul. 7, 2023.

(51) Int. Cl. A61K 35/30; A61K 38/46; A61P 25/28

(52) U.S. Cl. CPC A61K 35/30; A61K 38/465; A61P 25/28

(57) ABSTRACT

Disclosed herein are methods of treating ALS in a subject by administering a therapeutically effective amount of a composition comprising EVs, e.g., derived from neural cells, e.g., neural progenitor cells, to the subject. The EVs can be administered distally or peripherally to the CNS, such that the EVs cross the blood brain barrier and exert their therapeutic function in the CNS. The methods can reduce inflammation (e.g., NLRP3 inflammatory pathway signaling), reduce disease activity or progression, and/or improve motor or neurological performance, signs or symptoms associated with ALS, or survival in the ALS subject as compared to a control ALS subject. The methods of inhibiting necroptosis in a cell by contacting the cell with a therapeutically effective amount of a composition comprising EVs are also provided.

### RELATED APPLICATIONS

This application is a continuation application of International Application No. PCT/IB2024/056590, filed Jul. 5, 2024, which claims priority to U.S. Provisional Application No. 63/512,483, filed Jul. 7, 2023. The entire contents of each of the foregoing priority applications are incorporated by reference herein.

### BACKGROUND

Amyotrophic lateral sclerosis (ALS) is a fatal adult-onset neurodegenerative disease characterized by the selective degeneration of both upper and lower motor neurons progressive atrophy of muscle fibers and degeneration of upper and lower motor neurons. ALS spreads throughout the motor system, causing neuron loss at all levels of the motor system, from the cortex to the anterior horn of the spinal cord. ALS is a fatal disease, with a median survival period of 3 years from onset of weakness. Aspiration pneumonia, respiratory muscle atrophy, and medical complications of immobility contribute to morbidity in patients with the disease.

The cause of ALS is unknown, although a family history of the disease is obtained in about 5% of patients, and twin studies show a genetic contribution with heritability of about 61%. In some cases, ALS overlaps clinically, pathologically, and biologically with frontotemporal dementia, and it may share common biologic mechanisms with Alzheimer's disease, Parkinson's disease, and other neurodegenerative diseases.

Currently, there is no cure for ALS, with treatment options being directed to slowing progression of symptoms and preventing complications and sustaining activity of daily living of the patient. For example, the FDA-approved medications of ALS, riluzole, edaravone, and sodium phenylbutyrate-taurursodiol, reduce the rate of functional decline in ALS patients, and only slow disease progression by 2-3 months. Thus, more therapeutic options that can effectively treat ALS are critically needed.

### SUMMARY

Provided herein are compositions and methods relating to extracellular vesicles (EVs) and their use for treating ALS in a subject and reducing necroptosis and/or inflammation.

In a first aspect, provided herein is a method of treating amyotrophic lateral sclerosis (ALS) in a subject. The method comprises administering a therapeutically effective amount of a composition comprising extracellular vesicles (EVs) derived from neural cells to the subject, thereby treating ALS.

In some embodiments, the composition is administered to a site distal to the central nervous system (CNS) of the subject. In some embodiments, the composition is administered intravenously, intranasally, intraperitoneally, orally, or by combination of any thereof.

In some embodiments, neurological performance, motor performance, and/or one or more signs or symptoms associated with ALS are improved, and/or survival is prolonged in the subject relative to a control subject. In some embodiments, the one or more signs or symptoms comprise muscle weakness, walking difficulty, tripping, falling, hand weakness, hand clumsiness, slurred speech, chewing difficulty, swallowing difficulty, and/or breathing difficulty.

In some embodiments, level or activity of neurofilament light chain, chitinase-3-like protein 1 (CHI3L1), nucleotide-binding domain leucine-rich repeat containing family pyrin domain containing 3 (NLRP3), a NLRP3 signaling pathway molecule, and/or a proinflammatory cytokine is decreased in the subject. In some embodiments, the NLRP3 signaling pathway molecule comprises caspase-1, IL1-β, and/or IL-18. In some embodiments, the proinflammatory cytokine comprises TNFα and/or IL1-β.

In a second aspect, provided herein is a method of inhibiting necroptosis in a cell. The method comprises contacting the cell with a therapeutically effective amount of a composition comprising extracellular vesicles (EVs) derived from neural cells, thereby inhibiting necroptosis.

In some embodiments, level of receptor interacting protein kinase 1 (p-RIPK1) is decreased in the cell relative to a control cell.

In some embodiments, the cell is within a subject. In some embodiments, the subject has amyotrophic lateral sclerosis (ALS), and wherein neurological performance, motor performance, and/or one or more signs or symptoms associated with ALS are improved, and/or survival is prolonged in the subject relative to a control subject.

In some embodiments of the methods provided herein, the neural cells are non-transformed neural progenitor cells. In some embodiments, the neural cells are derived from human pluripotent stem cells, human induced pluripotent stem cells, or human embryonic stem cells.

In some embodiments, the EVs comprise ecto-5′-nucleotidase (CD73). In some embodiments, the EVs further comprise one or more of melanoma-associated chondroitin sulfate proteoglycan (MCSP), pentraxin-3 (PTX3), angiopoietin-1, insulin-like growth factor-binding protein 2 (IGFBP2), and macrophage colony-stimulating factor.

In some embodiments, the composition comprises about 1 mg to about 750 mg of EVs. In some embodiments, the EVs comprise exosomes. In some embodiments, the EVs are about 10 nm to about 10 μmin size, such as about 20 nm to about 250 nm in size.

In some embodiments, the EVs further comprise cargo. In some embodiments, the cargo comprises one or more of a nucleotide, a peptide, a protein, an antibody, and a small molecule. In some embodiments, the nucleotide is one or more of a siRNA, an antisense oligonucleotide, an mRNA, a plasmid, and a cDNA.

### BRIEF DESCRIPTION OF THE DRAWING

FIG. 1 is a graph showing phosphorylated receptor-interacting protein kinase 1 (p-RIPK1): GAPDH ratios in the HMC3 cells treated with TNFα in the presence of vehicle or EVs derived from neural progenitor cells provided herein.

FIG. 2 is a graph of neurological deficit score in SOD1-G93A mice (ALS mouse model) administered a weekly dose of the EVs or vehicle at symptom onset. * indicates p<0.05.

FIG. 3 is a graph of latency to fall in SOD1-G93A mice (ALS mouse model) administered a weekly dose of the EVs or vehicle at symptom onset. * indicates significance at p<0.05.

FIG. 4 is an overall survival curve of SOD1-G93A mice (ALS mouse model) administered a weekly dose of the EVs or vehicle at symptom onset. * indicates significance at p <0.05.

FIG. 5 is a graph of neurofilament light chain (NFL) levels in the serum of SOD1-G93A mice (ALS mouse model) administered a weekly dose of the EVs or vehicle at symptom onset. * indicates p<0.05. * indicates significance at p<0.05.

FIGS. 6A-6E depict a graph of levels of NLRP3 inflammasome pathway markers NLRP3 (FIG. 6A), caspase-1 (FIG. 6B), IL1-β (FIG. 6C), IL-18 (FIG. 6D), and an inflammatory marker TNFα (FIG. 6E) in the serum of SOD1-G93A mice (ALS mouse model) administered a weekly dose of the EVs or vehicle at symptom onset. * indicates p<0.05. * indicates significance at p<0.05.\

FIG. 7 is a graph of chitinase-3-like protein 1(CHI3L1 ) levels in the serum of SOD1-G93A mice (ALS mouse model) at day 80 of age. The mice were administered the EVs or vehicle 5 times a week beginning at day 69 of age. *** indicates p=0.0002.

FIG. 8 schematically depicts a potential mechanism by which the EVs provided herein ameliorates ALS-induced inflammation.

### DETAILED DESCRIPTION

### I. Definitions

In order that the present disclosure may be more readily understood, certain terms are first defined. In addition, it should be noted that whenever a value or range of values of a parameter are recited, it is intended that values and ranges intermediate to the recited values are also part of this disclosure. The term “blood brain barrier” as used herein refers to a highly selective semipermeable border of endothelial cells that prevents molecules in the circulating blood from nonselectively crossing into the extracellular fluid of the CNS. The blood brain barrier comprises endothelial cells of the capillary wall, astrocyte end-feet ensheathing the capillary, and pericytes embedded in the capillary basement membrane. The blood brain barrier prevents a variety of peripherally administered exogenous molecules, including non-neural EVs, from reaching the CNS to achieve a physiologically significant concentration in the CNS.

As used herein, the term “cargo” refers to one or more molecules that can be delivered to a target location in the body (e.g., the nervous system). Cargo can include, by way of example and without limitation, a small molecule (e.g., a small molecule drug), a nucleic acid (e.g., mRNA, siRNA. shRNA, antisense RNA, antisense oligonucleotide, miRNA, plasmid, cDNA, DNA), or a protein or peptide (e.g., a hormone, a growth factor, an enzyme, an anticoagulant, an interferon, an interleukin, an antibody, an antibody fragment, an antibody-drug conjugate, etc.). In some embodiments, the cargo is a therapeutic molecule. In some embodiments, the cargo can be attached directly or indirectly to the EV. In other embodiments, the cargo is delivered using an EV, as provided herein. As used herein, the term “central nervous system” or “CNS” refers to all structures within the

dura matter. Such structures include, but are not limited to, the cells and tissue of the brain and spinal cord. The CNS also comprises the cerebrospinal fluid, which fills the ventricles of the brain and the central canal of the spinal cord.

As used herein, the term “distal” or “peripheral” in the context of administration of a composition described herein refers to a site or route of administration that is distinct from a specified target location. For example, administration to a site distal to the brain refers to administration to a site other than the brain, e.g., intrathecal administration, intravenous administration, intranasal administration, etc., and can be contrasted with local administration to a site within the brain, e.g., via direct injection to the brain and surrounding parenchyma. In some embodiments, a composition comprising EVs described herein can be administered to a site distal to the brain. In other embodiments, a composition comprising EVs described herein can be administered to a site distal to the CNS. For example, routes of administration distal to the CNS can include intravenous administration and intranasal administration. Other suitable routes of administration, including those that are distal to the brain and/or distal to the CNS, are described herein.

The term “intact”, in the context of “intact blood brain barrier”, refers to a normally functioning blood brain barrier such that the blood brain barrier maintains a normal physiological function to nonselectively prevent molecules in the circulating blood from crossing into the extracellular fluid of the CNS and from being delivered to the brain. A subject comprising an intact blood brain barrier does not mean a healthy or non-diseased subject, e.g., not having a neurological condition or disorder (e.g., ALS). A subject may simultaneously have an intact blood brain barrier and a disease or condition, e.g., a neurological disorder, e.g., ALS. As used herein, the term “effective amount” or “therapeutically effective amount” refers to the amount of an agent, e.g., a composition containing EVs,, which is sufficient to reduce or ameliorate the severity and/or duration of a disorder, or one or more symptoms thereof, prevent the advancement of a disorder, cause regression of a disorder, prevent the recurrence, development, onset, or progression of one or more symptoms associated with a disorder, or enhance or improve the prophylactic or therapeutic effect(s) of another therapy. For example, an effective amount of an EV composition for inhibiting necroptosis or reducing inflammation can be an amount sufficient to inhibit necroptosis or reduce inflammation in a cell of a subject being contacted with the EV composition, e.g., as measured by levels of markers for necroptosis or inflammation (e.g., p-RIPK1, NLRP3, caspase-1, IL-1β, IL-18, TNFα). For another example, an effective amount of an EV composition for treating ALS can be an amount sufficient to slow progression of ALS, maintain neurological performance or motor performance, improve one or more signs or symptoms associated with ALS (e.g., muscle weakness, walking difficulty, tripping, falling, hand weakness, hand clumsiness, slurred speech, chewing difficulty, swallowing difficulty, and/or breathing difficulty), and/or improve survival in the subject. For example, if a given clinical treatment is considered effective when there is at least a certain percentage of reduction in a measurable parameter associated with ALS, a therapeutically effective amount of a composition (e.g., EV composition) for the treatment of ALS is the amount necessary to obtain at least the certain percentage of reduction in that parameter. One skilled in the art can select the specific percentage, or range of percentages, of reduction of a measurable parameter by which to consider a treatment as effective according to the clinical and scientific context. The term “embryonic stem cell” or “ESC” refers to pluripotent cells, preferably of primates, including humans, which are isolated from the blastocyst stage embryo.

The terms “extracellular vesicle” and “EV” are used herein to refer to a vesicle of about 10 nm to 10 μm in size that is enclosed by a lipid bilayer, e.g., a portion of a cell membrane. EVs can contain fluid, macro-molecules, solutes, and metabolites from a cell. The term “EV” also includes lipid vesicles engineered to contain bioactive molecules found in a cell-derived EV, such as a neural EV. These terms encompass, but are not limited to, a microvesicle (e.g. any vesicle shed from the plasma membrane of a cell), an exosome (e.g. any vesicle derived from the endo-lysosomal pathway), an apoptotic body (e.g. obtainable from apoptotic cells), a microparticle (which may be derived from e.g. platelets), an ectosome (derivable from e.g. neutrophils and monocytes in serum), prostasome (e.g. obtainable from prostate cancer cells), or a cardiosome (e.g. derivable from cardiac cells). Furthermore, EVs may refer to extracellular vesicle mimics or cellular membrane vesicles obtained through membrane extrusion or other techniques. The term “EV” encompasses vesicles that have been isolated from a recombinant cell source engineered or modified to produce/express an exogenous agent (e.g., an exogenous polypeptide), which agent is incorporated into the EV. The term “EV” also encompasses vesicles that have been engineered (e.g., loaded) following isolation to contain an exogenous agent (e.g., an exogenous polypeptide). The term “EV” also includes artificial lipid vesicles engineered to contain bioactive molecules found in a cell-derived EVs, such as a neural EVs. The term EV encompasses both exosomes and ectosomes.

EVs may be obtained from the appropriate biological source using a combination of isolation techniques, for example, centrifugation, filtration and ultracentrifugation methodologies. Exosomes are released on the exocytosis of multivesicular bodies (MVBs). Ectosomes are vesicles assembled at and released from the plasma membrane. In some cases, the EV is about 20 nm to 10 μm, 20 nm to 1 μm, 20 nm to 500 nm, 30 nm to 100 nm, 30 nm to 160 nm, or 80 nm to 160 nm in size. In some embodiments, the EVs are exosomes that are about 20 nm to 150 nm in size. EVs may be isolated from any suitable biological sample from a mammal, including but not limited to, whole blood, serum, plasma, breast milk, cerebrospinal fluid, amniotic fluid, ascitic fluid, or bone marrow. EVs can be isolated from cultured mammalian cells (e.g., neural cells, dendritic cells (wild-type or immortalized), induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes and the like). In particular embodiments, EVs can be isolated from a neural cell (e.g., a neural progenitor cell, a neural stem cell, a glial cell, an astrocyte, a neuron, etc.). The term “pluripotent stem cells” (PSCs), of which “mbryonic stem cells” (ESCs) and induced pluripotent stem cells (iPSCs) are a subset, are derived from pre-embryonic, embryonic, fetal tissue or adult stem cells (in the case of induced pluripotent stem cells) at any time after fertilization, and have the characteristic of being capable under appropriate conditions of producing progeny of several different cell types, especially including neuronal stem cells and progenitors, neural crest cells, mesenchymal stem cells (MSCs) and related proliferative and non-proliferative neural cells. The term includes both established

lines of stem cells of various kinds, and cells obtained from primary tissue that are pluripotent in the manner described. Human pluripotent stem cells include, for example, human embryonic stem cells (hESCs). Human pluripotent stem cells also can include pluripotent stem cells derived from adult cells, such as induced pluripotent stem cells (hiPSCs). “Pharmaceutically acceptable” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. In some instances, “pharmaceutically acceptable” refers to those compounds that are suitable for use in contact with the tissues of human beings and animals without excessive toxicity, irritation, allergic response, immunogenicity, or complications commensurate with a reasonable benefit/risk ratio.

As used herein, the term “neural cell” refers to a cell pertaining to the nervous system. The term “neural cell” encompasses neural stem cells, e.g., neural progenitor (NP) cells, and differentiated neural cells. In some embodiments, differentiated neural cells can be derived in vitro from neural stem cells, NP cells or from pluripotent stem cells. Exemplary neural cells include a neuron, a glial cell, an astrocyte, an oligodendrocyte, a microglial cell, a Schwann cell, and a glioma cell. In other embodiments, the neural cell can be a neural progenitor (NP) cell or a neural stem cell. The terms “neural progenitor cell” and “neural stem cell” refer to multipotent cells that have the capacity to differentiate into a restricted repertoire of neuronal and glial cell types. In some embodiments, NP cells can be derived in vitro from pluripotent stem cells, e.g., induced pluripotent stem cells (iPS cells) or embryonic stem cells (ES cells). In some embodiments, the neural cells referenced herein are human neural cells. In exemplary embodiments, the NP cells referenced herein are human NP cells. The NP cells can be non-transformed. “Transformed” as used herein refers to a cell having a genetic alteration resulting from the incorporation of exogenous genetic material. “Non-transformed” as used herein refers to a cell not having a genetic alteration or an incorporation of exogenous genetic material. Transformation of a cell can result in material changes in the compositions or characteristics of EVs derived from the cell. In some embodiments, the NP cells are proliferative. In some embodiments, the NP cells maintain phenotype without differentiation.

The term “neural EV,” “neural cell EV,” or “neural cell derived EV” is used to refer to an EV that is derived from neural cells, for example, neural stem cells, e.g., neural progenitor cells. A “neural EV” can be a “neural progenitor EV” or an EV derived from other types of neural cells, such as a neuron, a glial cell, an astrocyte, an oligodendrocyte, a microglial cell, a Schwann cell, or a glioma cell, or a progenitor cell thereof. The term also refers to vesicles engineered to contain a sufficient number of the bioactive molecules found in cell-derived neural EV to have substantially the same bioactivity.

The term “neural progenitor EV,” “neural progenitor cell EV,” or “neural progenitor cell derived EV” is used interchangeably to refer to an EV that is derived from neural progenitor cells. The term also refers to vesicles engineered to contain a sufficient number of the bioactive molecules found in cell-derived neural progenitor EV to have substantially the same bioactivity.

“Pharmaceutically acceptable” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. In some instances, a “pharmaceutically acceptable” refers to those compounds that are suitable for use in contact with the tissues of human beings and animals without excessive toxicity, irritation, allergic response, or complications commensurate with a reasonable benefit/risk ratio.

As used herein, the term “sample” refers to a specimen (e.g., cell (e.g., neural cell), tissue (e.g., spinal cord, brain), blood, blood component (e.g., serum or plasma), urine, saliva, amniotic fluid, cerebrospinal fluid, pancreatic fluid, chorionic villus sample, and bone marrow). In some embodiments, a sample is derived from or taken from a subject.

As used herein, the term “subject” refers to any organism that is the target of administration or treatment. A “subject” can be an organism, for example, a mammal (e.g., a human, a non-human mammal, a non-human primate, a primate, a laboratory animal, a mouse, a rat, a hamster, a cat, or a dog). In one embodiment, a subject is a human subject. The term “patient” refers to a human subject who has been diagnosed with a disease or disorder, including ALS, a human subject who shows one or more symptoms of any of the disorders or diseases (e.g., ALS) described herein, and/or human subject under the treatment of a clinician, e.g., physician. A subject can be male or female.

In some embodiments of the present disclosure, subjects are in need of delivery of EVs to the CNS. In some embodiments, subjects are in need of delivery of exogenous cargo loaded in EVs to the CNS. In some embodiments, subjects are in need of treatment of ALS, or reduction of inflammation in the brain. In some embodiments, subjects have a neurological condition or a symptom associated with ALS.

The term “treat” and “treatment” refers to the medical management of a subject with the intent to improve, ameliorate, stabilize (e.g., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. Treatment does not require the complete amelioration of a symptom or disease and encompasses embodiments in which one reduces symptoms and/or underlying risk factors. “Treatment” can also mean prolonging survival as compared to expected survival if not receiving treatment. Those in need of treatment include those already with the condition or disorder, as well as

those prone to have the condition or disorder or those in which the condition or disorder is to be prevented. The term “prevent” does not require the 100% elimination of the possibility of an event. Rather, it denotes that the likelihood of the occurrence of the event has been reduced in the presence of the compound or method.

In accordance with the present invention there may be employed conventional cell culture methods, chemical or synthetic methods, and other biological and pharmaceutical techniques within the skill of the art. Such techniques are well-known and are otherwise explained fully in the literature. Standard techniques for growing cells, separating cells, and where relevant, cloning, DNA isolation, amplification and purification, for enzymatic reactions involving DNA ligase, DNA polymerase, restriction endonucleases and the like, and various separation techniques can include those known and commonly employed by those skilled in the art. A number of standard techniques are described in Sambrook et al., 1989 Molecular Cloning, Second Edition, Cold Spring Harbor Laboratory, Plainview, New York; Maniatis et al., 1982 Molecular Cloning, Cold Spring Harbor Laboratory, Plainview, New York; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, New York; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The term “antibody” is used herein in the broadest sense and encompasses various structures that bind a target antigen, including but not limited to monoclonal antibodies, polyclonal antibodies, multispecific antibodies (e.g., bispecific antibodies), nanobodies, monobodies, antibody mimetics, and antibody fragments so long as they exhibit the desired antigen-binding activity. Where a range of values is provided, it is understood that each intervening value, unless the context clearly dictates otherwise, between the upper and lower limit of that range and any other stated or intervening value in that stated range is encompassed within the invention as if explicitly stated. The upper and lower limits of these ranges may independently be included in the ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, exemplary methods and materials are now described.

As used herein and in the appended claims, the singular forms “a,” “an,” and “the” include plural references unless the context clearly dictates otherwise. For example, “an object” means one or more objects.

The term “about” or “approximately” usually means within 5%, or more preferably within 1%, of a given value or range.

### II. Extracellular Vesicles (EVs)

Extracellular vesicles (EVs), such as exosomes, can be used to enhance the efficiency of nucleic acid delivery. Exosomes are small membrane-bound vesicles of endocytic origin that are released into the extracellular environment following fusion of multivesicular bodies with the plasma membrane. Exosome production has been described from many types of cells, including immune cells such as B cells, T cells, and dendritic cells (DCs); tumor cells; mesenchymal stem cells (MSCs); and more recently neural cells.

EVs comprise a heterogeneous group of small structures enclosed by a lipid bilayer, for example, a portion of a cellular plasma membrane. EVs can range in size from about 10 nm to 10 μm in diameter, and most commonly fall within the range of about 25-500 nm. EVs can be broadly divided into two classes, exosomes and ectosomes. Exosomes can be formed by cells through the inward budding of the endosomal membrane during the maturation of multivesicular bodies (MVBs). Exosomes can then be released to the extracellular space by fusion of MVBs with the cell surface. Exosomes are typically 25-2500 nm in diameter, and in some embodiments, can be within the range of about 25-200 nm, about 30-150 nm, or about 50-200 nm. Ectosomes, also known as microvesicles, can be formed by cells through budding of the plasma membrane. Ectosomes can vary in size from about 10 nm to 10 μm, and in some embodiments, can be within the range of about 10-1000 nm, about 10-500 nm, about 10-250 nm, or about 10-200 nm.

EVs are involved in intercellular communication, allowing for the transfer of material from EVs to cells by fusion with the cell membrane or via cellular uptake, including, but not limited to caveolin-independent and-dependent mechanisms, clathrin-independent and -dependent mechanisms, macropinocytosis and/or phagocytosis. EVs have been reported to be involved in numerous physiological processes, including immune modulation, angiogenesis, migration of endothelial cells in connection with tumor growth, or reducing damage in ischemia reperfusion injury. Many of these functions are mediated by nucleic acids, proteins, or lipids contained in or on the vesicles.

EVs can carry cargo in the lumen and/or embedded in or attached to the lipid bilayer. Cargo carried by EVs can include proteins, lipids, and/or nucleic acids, such as, mRNA or miRNA. In cases where EVs are produced by cells, the composition of the cargo is highly dependent on the cell type. For example, it has been shown that EVs derived from astrocytes, neural progenitor cells, and mesenchymal stem cells each contain a distinct complement of protein cargo (see, U.S. Patent Application Publication No. US2018/0327714A1, the contents of which are incorporated herein by reference in its entirety). EVs derived from these different cell types also contain a distinct profile of nucleic acid molecules, including mRNA and/or miRNA. In embodiments where EVs are obtained from cells, the EVs contain endogenous cargo that reflects the contents of EVs produced by the cells from which the EVs are derived. In some embodiments, EVs obtained from cells can also contain exogenous cargo. Exogenous cargo includes proteins, nucleic acids, small molecules, or lipids that are introduced into EVs by manipulation of the vesicles following their release from production cells. In other embodiments, EVs obtained from cells can contain exogenous cargo, which is packaged into EVs as a result of a recombinant nucleic acid present in the cells from which the EVs are derived. For example, EVs containing a recombinant protein can be derived from cells that contain a recombinant nucleic acid encoding the protein. In embodiments where EVs are produced synthetically, suitable cargo can be selected for inclusion in the vesicles. For example, in one embodiment, synthetic EVs can contain one or more proteins, lipids, or nucleic acids present in EVs derived from neural cells, e.g., neural progenitor cells, neurons, glial cells, or astrocytes. EVs suitable for use in the compositions and methods described herein can be derived from any suitable source. For example, EVs can be derived from neural cells, such as neural stem cells, neural progenitor cells, or differentiated neural cells, such as neurons, glial cells, or astrocytes. EVs suitable for use in the methods described herein can also be produced synthetically. In some embodiments, EVs used in the compositions and methods described herein are derived from neural progenitor cells or neural stem cells. Neural EVs, including neural progenitor EVs, contain a complement of membrane proteins that, without wishing to be bound by theory, are believed to facilitate passage of neural EVs across the capillaries of the blood brain barrier. Conventional methods of preparing EVs for nucleic acid delivery, such as electroporation, can compromise membrane integrity, and consequently a reduced capacity to traverse the blood brain barrier. Provided herein are neural EVs comprising exogenous nucleic acids, uses thereof in the treatment of neurological disease, and methods of loading EVs with exogenous nucleic acid in a manner that preserves membrane integrity.

EVs (e.g., exosomes) derived from cells comprise a variety of biological molecules that reflect their cellular origin. In one embodiment, provided herein is a population of EVs derived from neural cells, e.g., neural progenitor cells. Such neural EVs contain a milieu of different proteins, including cytokines and growth factors, and coding and noncoding RNA molecules, derived from neural cells. The cargo contained in neural EVs can impact neural and vascular function by providing neuroprotection, reducing inflammation, immunomodulatory via acting on T cells macrophages and microglia, reducing oxidative stress, improving vascular integrity, impacting metabolic activity, and inducing a neuroregenerative effect via an increase in neurogenesis, cell migration, re-myelination and differentiation. Without wishing to be bound by theory, the combination of native proteins expressed on the surface of EVs derived from neural cells (e.g., neural progenitor cells, neural stem cells) is thought to allow the vesicles to localize to the CNS and traverse the blood brain barrier to a greater extent than EVs derived from other sources. Moreover, the ability of neural EVs to target the CNS and/or cross the blood brain barrier is not impeded by loading the EVS with nucleic acids. In some embodiments of the present disclosure, cargo-loaded EVs, which include EVs derived from neural cells (e.g., neural progenitor cells, neural stem cells), are able to transport functionally intact cargo across the blood brain barrier for delivery to targets in the brain and central nervous system.

In some embodiments, neural cell derived EVs, e.g., exosomes, can contain membrane proteins, including but not limited to CD63, CD81, and CD133. Accordingly, in one embodiment, the EVs, e.g., exosomes, suitable for use in the compositions and methods described herein include one or more (i.e., one or more, two or more, or all three) of the following cell surface proteins: CD63, CD81, and CD133. In addition, the EVs, e.g., exosomes, can include one or more (i.e., one or more, two or more, three or more, four or more, five or more, six or more, seven or more, eight or more, nine or more, ten or more, eleven or more, or all twelve) of the following miRNAs: hsa-miR-135a-2, hsa-miR-124-1, hsa-miR-124-2, hsa-miR-124-3, hsa-miR-489, hsa-miR-9-3, hsa-miR-9-2, hsa-miR-9-1, hsa-miR-219b, hsa-miR-219a-2, hsa-miR-363 and/or hsa-miR-20b. In one embodiment, the isolated population of EVs are enriched for one or more of the foregoing miRNAs. Enrichment can be measured in absolute or relative quantities, such as when compared to unmodified exosomes derived from non-neural cells, e.g., mesenchymal stem cells. As described herein, neural progenitor cell derived EVs (NP EVs) carry a number of miRNAs that are enriched in the vesicles relative to the parent NP cells, or to EVs derived from other cell sources. Accordingly, in some embodiments, EVs derived from neural progenitor cells can comprise one or more of the following miRNAs: miR-92a, miR-219a-2, miR-363, miR-21, miR-124, miR-135a-2, miR-130b, miR-148a, miR-25, miR-30a-5p, miR-30a-3p, miR-340, miR-122, and/or miR-31-5p. In some embodiments, the NP EVs can comprise 1, 2, 3, 4, 5, 6, 7, 8, 9, 10, 11, 12, 13, or 14 of the foregoing miRNAs. In some embodiments, the NP EVs comprise one, two, three, or four of miR-219a-2, miR-363, miR-124, miR-135a-2, or combinations thereof. In some embodiments, the NP EVs comprise miR30a-3p and/or miR-340. In some embodiments, the NP EVs comprise miR-122 and/or miR-31-5p. In some embodiments, the NP EVs comprise one, two, three, four, five, six, seven, or eight of miR-219a-2, miR-363, miR-124, miR-135a-2, miR30a-3p, miR-340, miR-122, miR-31-5p, or combinations thereof.

### (i) Therapeutic Effects of EVs

EVs (e.g., neural EVs, neural progenitor EVs) provided herein can exert therapeutic activity, such as anti-inflammatory effects, with no additional payload (e.g., exogenous cargo). For example, in some embodiments, the neural EVs (e.g., neural progenitor EVs) provided herein comprise ecto-5′-nucleotidase (NT5E). NT5E, also referred to as AMPase and CD73, converts AMP to adenosine. Without wishing to be bound by theory, during neurodegeneration, after stroke, and upon other neural insults, dead and dying cells release high amounts of ATP into the extracellular space. ATP is a potent inflammatory signal, causing secondary injury in post-acute phase, while suppressing and/or killing the anti-inflammatory regulatory T cells. The NT5E activity confers the EVs the ability to convert ATP, which is pro-inflammatory, into adenosine, which is anti-inflammatory and supports immunosuppressive environment. NT5E (CD73) can be a potency marker for EVs. In further embodiments, the neural EVs provided herein comprise other biologically active polypeptides or proteins, such as melanoma-associated chondroitin sulfate proteoglycan (MCSP), pentraxin-3 (PTX3), angiopoietin-1, insulin-like growth factor-binding protein 2(IGFBP 2 ), and macrophage colony-stimulating factor. The EVs (e.g., neural EVs, neural progenitor EVs) provided herein can cross the blood-brain barrier (including the intact blood brain barrier) and exert therapeutic activity without additional payload. The EVs can reduce neuro-inflammation, including reducing pro-inflammatory T cells and increasing anti0inflammatory T cells and anti-inflammatory macrophages (Webb, R.L., et. al. 2018 Transl Stroke Res. 9(5): 530-539). Additionally, or alternatively, the EVs can promote neuro-protection, including preventing cell death by reducing oxidative stress to enhance neural survival (Webb, R. L. 2018 Stroke 49(5): 1248-1256; Webb, R.L., et. al. 2018 Transl Stroke Res 9(5): 530-539). Further, the EVs can stimulate neuro-regeneration, including increasing neural stem cell proliferation resulting in neural growth and remyelination (Sun, M., et. al. 2020 Journal of Neurotrauma, doi.org/10.1089/neu.2019.6443).

### (ii) EV Production

EVs suitable for use in compositions and methods disclosed herein can be produced by a variety of cell types (e.g., human neural progenitor cells, human neural stem cells, murine neural progenitor cells, murine neural stem cells, etc.), or can be produced synthetically. EVs can be purified from any cell source, as described above (e.g., cultured mammalian cells, including but not limited to primary cells, stem/progenitor cells, transformed cells, and established cell lines) using known methods. For example, EVs can be purified through a procedure selected from the group of techniques comprising liquid chromatography (LC), high-performance liquid chromatography (HPLC), spin filtration, tangential flow filtration, hollow fiber filtration, centrifugation, immunoprecipitation, flow field fractionation, dialysis, microfluidic-based separation, etc., or any combination thereof. In one embodiment, the purification of the EVs is carried out using a sequential combination of filtration (preferably ultrafiltration (UF), tangential flow filtration or hollow fiber filtration) and size exclusion liquid chromatography (LC). This combination of purification steps results in optimized purification, which in turn leads to superior therapeutic activity. Further, as compared to ultracentrifugation (UC), which is routinely employed for purifying EVs such as exosomes, sequential filtration-chromatography is considerably faster and possible to scale to higher manufacturing volumes, which is a significant drawback of the current UC methodology more commonly used. Another purification methodology is tangential flow filtration (TFF), which offers scalability and purity, and may be combined with other types of purification techniques such as filtration.

EVs suitable for use according to some embodiments of the present disclosure may be derived from neural cells, dendritic cells (wild-type or immortalized), induced and non-induced pluripotent stem cells, fibroblasts, platelets, neutrophils, monocytes, immune cells, reticulocytes, cardiac cells, tumor cells, mesenchymal stem cells, satellite cells, hematopoietic stem cells, pancreatic stem cells, white and beige pre-adipocytes, and apoptotic cells. In some embodiments, EVs are derived from cultured cell lines, e.g., CHO cells, HEK cells. In some embodiments, EVs are derived from primary cells. In some embodiments, EVs are derived from cells isolated from a subject and cultured to produce exosomes. In some embodiments, EVs can be derived from neural cells, such as neural progenitor cells, neurons, glial cells, astrocytes, oligodendrocytes, microglia, Schwann cells, or glioma cells. In some embodiments, the neural cells are non-transformed cells. In some embodiments, the neural cells are primary neural cell cultures. In some embodiments, EVs can be produced by transformed cell lines, e.g., transformed neural cell lines. In other instances, EVs can be produced by engineered cell lines, e.g., recombinant cell lines, that express exogenous polypeptides and/or nucleic acids. In a specific embodiment, EVs are neural EVs derived from neural cells, such as neural progenitor cells, or neural stem cells, neurons, oligodendrocytes, microglia, Schwann cells or astrocytes.

In some embodiments, the EVs comprise exosomes. Exosomes can be derived from any of the foregoing cell types. For example, exosomes suitable for use in the compositions and methods disclosed herein can be neural exosomes. Neural exosomes can be derived from neural cells, including but not limited to neural progenitor cells, neurons, astrocytes, oligodendrocytes, microglia, Schwann cells, or glioma cells. In other embodiments, the exosomes can be produced synthetically.

Alternatively, the EVs can be ectosomes, also known as microvesicles. Microvesicles can be derived from any of the foregoing cell types. For example, microvesicles suitable for use in the compositions and methods disclosed herein can be neural microvesicles. Neural microvesicles can be derived from neural cells, including but not limited to neural progenitor cells, neurons, astrocytes, oligodendrocytes, microglia, Schwann cells, or glioma cells. In other embodiments, the microvesicles can be produced synthetically.

In some embodiments, the compositions and methods described herein can employ an isolated and/or purified population of EVs that comprises exosomes and/or ectosomes.

In some instances, the disclosed EVs can be obtained by culturing cells, such as neural cells, for a time sufficient for the cells to produce EVs. Cells used to produce EVs can be derived, in some embodiments, from pluripotent stem cells, for example, human embryonic stem cells (hESCs) or induced pluripotent stem cells (iPSCs). EVs can be isolated from cell culture medium or tissue culture supernatant. EVs produced from cells can be collected from the culture medium by any suitable method. Typically, an isolated population of EVs can be prepared from cell culture or tissue supernatant by centrifugation, size exclusion columns, microfluidic devices, polymer precipitation, filtration or combinations of these methods, as described, for example, in U.S. Patent Application Publication No. 2014/0356382, which is hereby incorporated by reference in its entirety. For example, EVs can be prepared by differential centrifugation, that is low speed (<2,0000 g) centrifugation to pellet larger particles followed by high speed (>100,000 g) centrifugation to pellet EVs, size filtration with appropriate filters (for example, 0.22 μm filter), gradient ultracentrifugation (for example, with sucrose gradient), or a combination of these methods. In some embodiments, the EV-producing cells disclosed herein are cultured for about 1, 2, 3, 4, 5, 6, 7, 8, 9, 10, 11, 12, 13 or 14 days or for as long as about 1, 2, 3, 4, 5, 6, 7, 8 weeks or about 1, 2, 3, 4, 5, or 6 months, depending on the cell and its ability to produce EVs. The EV-producing cells may be cultured in suitable media and grown under conditions that are readily determined by one of ordinary skill in the art. Cell culture conditions may vary with cell type, and the examples presented hereinafter illustrate suitable media and conditions. For example, CMRL 1066 medium (from INVITROGEN) with exosome-depleted fetal bovine serum (e.g., at 10%) and optionally supplemented with glutamine or glutamine-containing mixtures and antibiotics could be used. Cells can be grown adhering on a surface in some embodiments, e.g. they can be grown as a monolayer to multilayers on the surface (feeder cell free) and may be grown until 30, 40, 50, 60, 70, 80, 90, 95 or 100% confluent. In other embodiments, the cells can be grown as cell aggregates or on microbeads in suspension cultures.

Cell growth media are well known in the art and generally comprise at least a minimum essential medium plus one or more optional components such as growth factors, ascorbic acid, glucose, non-essential amino acids, salts (including trace elements), glutamine, insulin (where indicated and not excluded), Activin A, transferrin, beta mercaptoethanol, and other agents well known in the art and as otherwise described herein. A preferred media for some embodiments is a low protein, serum-free growth medium that supports neural cells. The growth factor used can be fibroblast growth factor 2(FGF 2 ), alone or preferably in combination with leukemia inhibitor factor (LIF).

In embodiments in which NP or neural cells are to be grown in the growth media, the inclusion of LIF is preferred but may not be required. Additional suitable media types include basal cell media which may contain serum, for example, between about 0.1% and 20% (preferably, about 2-10%) fetal calf serum, or for defined medium, an absence of fetal calf serum and KSR, and optionally including bovine serum albumin (about 1-5%, preferably about 2%). In some instances, the medium is defined and is serum-free and has low protein content. In other instances, the media is media and supplement from ARUNA BIOMEDICAL, INC. (e.g., AB2™ Neural Cell Culture Media Kit from ARUNA BIOMEDICAL, INC. containing AB2™ Basal Neural Medium and ANS™ Neural Medium Supplement), which allow neural cultures to maintain a stable karyotype over multiple passages without the need for feeder cells, making them an excellent choice for a wide variety of research applications including early stage drug discovery. The components of the growth media depend on the type of neural cell to be grown, all of which are well known in the art. In one instance, a AB2™ Neural Cell Culture Media Kit is used and it contains AB2™ Basal Neural Medium and ANS™ Neural Medium Supplement. In a specific instance, the medium and supplement described in the instance above are specifically engineered for versatility to meet all neural cell culture needs. The AB2™ Basal Neural Medium and ANS™ Neural Medium Supplement can be used as the base for specialized mediums to direct differentiation of hNP cells (e.g., hNP1™ line) toward various neural phenotypes. Each lot of medium and supplement is pre-qualified for use by testing for cell growth, sterility, pH, osmolarity, and endotoxins.

Other agents which optionally may be added to the medium include, depending on the cell type grown in the media, for example, any one or more of nicotinamide, members of TGF-β family, including TGF-β 1, 2, and 3, Activin A, nodal, Bone Morphogen Proteins (BMP 2 to 7), serum albumin, members of the fibroblast growth factor (FGF) family, platelet-derived growth factor-AA, and-BB, platelet rich plasma, insulin growth factor (IGF-I, II, LR-IGF), growth differentiation factor (GDF-5,-6,-8,-10, 11), glucagon like peptide-I and II (GLP-I and II), GLP-1 and GLP-2 mimetobody, Exendin-4, parathyroid hormone, insulin, progesterone, aprotinin, hydrocortisone, ethanolamine, epidermal growth factor (EGF), gastrin I and II, copper chelators such as, for example, triethylene pentamine, forskolin, Na-Butyrate, betacellulin, ITS, noggin, neurite growth factor, nodal, valproic acid, trichostatin A, sodium butyrate, hepatocyte growth factor (HGF), sphingosine-1, VEGF, MG132 (EMD, CA), N2 and B27 supplements (Gibco, CA), steroid alkaloid such as, for example, cyclopamine (EMD, CA), keratinocyte growth factor (KGF), Dickkopf protein family, bovine pituitary extract, islet neogenesis-associated protein (INGAP), Indian hedgehog, sonic hedgehog, proteasome inhibitors, notch pathway inhibitors, sonic hedgehog inhibitors, heregulin, or combinations thereof, among a number of other components. Each of these components, when included, are included in effective amounts.

In some instances, suitable media may be made from the following components, such as, for example, Dulbecco's modified Eagle's medium (DMEM), GIBCO #11965-092; Knockout Dulbecco's modified Eagle's medium (KO DMEM), GIBCO #10829-018; Ham's F12/50% DMEM basal medium; 200 mM L-glutamine, GIBCO #15039-027; non-essential amino acid solution, GIBCO 11140-050; β-mercaptoethanol, SIGMA #M7522; human recombinant basic fibroblast growth factor (bFGF), GIBCO #13256-029. Other suitable reagents can include Neurobasal (GIBCO), BrainPhys (STEM CELL TECHNOLOGIES) and/or NeuroDiff (STEM CELL TECHNOLOGIES).

Cell media are commercially available and can be supplemented with commercially available components, including defined xeno-free components, such as those available from INVITROGEN CORP. (GIBCO), CELL APPLICATIONS, INC., BIOLOGICAL INDUSTRIES, BETH HAEMEK, Israel, and CALBIOCHEM. One of ordinary skill in the art will be able to readily modify the cell media to produce any one or more of the target cell types to be used as a source of EVs.

The disclosed EV-producing cells may be cultured on a layer of feeder cells that support the cells in various ways. Approaches for culturing cells on a layer of feeder cells are well known in the art. The cells may be grown on a cellular support or matrix, as adherent monolayers, or cell aggregates in suspension. In some instances, the use of a cellular support may be preferred, depending upon the cells used to produce the EVs. When used, cellular supports preferably comprise at least one substrate protein. Substrate proteins include, for example, an extracellular matrix protein, which is a protein found in the extracellular matrix, such as laminin, tenascin, thrombospondin, and mixtures thereof, which exhibit growth promoting and contain domains with homology to epidermal growth factor (EGF) and exhibit growth promoting activity. Other substrate proteins which may be used include for example, collagen, fibronectin, vibronectin, polylysine, polyornithine and mixtures thereof. In addition, gels and other materials such as methylcellulose of other gels which contain effective concentrations of one or more of these embryonic stem cell differentiation proteins may also be used. Exemplary differentiation proteins or materials which include these differentiation proteins include, for example, recombinant laminin, BD Cell-Tak™ Cell and Tissue Adhesive, BD™ FIBROGEN Human Recombinant Collagen I, BD™ FIBROGEN Human Recombinant Collagen III, BD Matrigel™ Basement Membrane Matrix, BD Matrigel™ Basement Membrane Matrix High Concentration (HC), BD™ PuraMatrix™ Peptide Hydrogel, Collagen I, Collagen I High Concentration (HC), Collagen II (Bovine), Collagen III, Collagen IV, Collagen V, and Collagen VI, among others.

Alternatively, these cells may be cultured in a culture system that is free of feeder cells, or essentially free of feeder cells, but nonetheless supports proliferation of the cells to produce EVs. The growth of cells in feeder-free culture can be supported using a medium conditioned by culturing previously with another cell type. Alternatively, the growth of EV-producing cells in feeder-free culture without differentiation can be supported using a chemically defined medium. These approaches are well known in the art. In certain embodiments of the present invention, the cells are grown in feeder cell free medium.

In some embodiments of the present disclosure, EVs can be isolated from neural cells, e.g., neural progenitor cells, that are derived from (e.g., have differentiated from) pluripotent stem cells, such as hESCs or iPSCs. Pluripotent stem cells may express one or more of the stage-specific embryonic antigens (SSEA) 3 and 4, and markers detectable using antibodies designated Tra-1-60 and Tra-1-81, as described by Thomson et al. (Science 282:1145, 1998). Differentiation of pluripotent stem cells in vitro results in the loss of SSEA-4, Tra-1-60, and Tra-1-81 expression (if present) and increased expression of SSEA-1. Undifferentiated pluripotent stem cells typically have alkaline phosphatase activity, which can be detected by fixing the cells with 4% paraformaldehyde, and then developing with Vector Red as a substrate, as described by the manufacturer (VECTOR LABORATORIES, Burlingame Calif.). Undifferentiated pluripotent stem cells also typically express Oct-4 and TERT, as detected by antibodies or RT-PCR. In some embodiments, EVs for use in the compositions and methods described herein can be produced by neural progenitor cells derived from hESCs. In other embodiments, EVs for use in the compositions and methods described herein can be produced by neural progenitor cells derived from iPSCs.

Types of pluripotent stem cells that may be used for generation of EVs include established lines of pluripotent cells derived from tissue formed after fertilization, including pre-embryonic tissue (such as, for example, a blastocyst), embryonic tissue, or fetal tissue taken any time during gestation, typically but not necessarily before approximately 10-12 weeks gestation. Non-limiting examples are established ethical lines of hESCs or human embryonic germ cells, such as, for example the hESC lines WA01, WA07, and WA09 (WICELL). Also contemplated herein is the use of pluripotent stem cells during initial establishment or stabilization of such cells, in which case the source cells would be primary pluripotent cells taken directly from the source tissues. Also suitable are cells taken from a pluripotent stem cell population already cultured in the absence of feeder cells. Also suitable are mutant hESC lines, such as, for example, BG01v (VIACYTE, San Diego, CA), as well as normal hESC lines, such as WA01, WA07, WA09 (WICELL, Madison, WI) and BG01, BG02 (VIACYTE, San Diego, CA).

hESCs may be prepared by methods described in the art, e.g., by Thomson et al. (U.S. Pat. No. 5,843,780; Science 282:1145-1147, 1998; Curr Top Dev Biol 38:133-165, 1998; Proc Natl Acad Sci 92:7844-7848, 1995). Alternatively, they may be obtained commercially.

iPSCs are made by dedifferentiating adult somatic cells back to a pluripotent state. iPSCs can be generated by any suitable methodology, including, but not limited to by artificial expression of four genes, c-myc, Klf4, SOX2 and OCT4, or similar.

Methods for the production of human neural progenitor (hNP) cells from hESCs are described, for example, in U.S. Pat. No. 7,531,354, which is hereby incorporated by reference in its entirety. hNPs can express markers associated with the earliest multipotent neural stem cells, including, for example, Nestin, Musashi-1, SOX1, SOX2 and SOX3. In one instance, the hNPs express SOX1 (SOX1⁺). In another instance, the hNPs express SOX2 (SOX2+). In some other instances, the hNPs express SOX3 (SOX3⁺). In some specific instances, the hNPs express at least one of Nestin, Musashi-1, SOX1, SOX2 and SOX3. In other instances, the hNPs express two or more of Nestin, Musashi-1, SOX1, SOX2 and SOX3. In yet another instance, the hNPs express three or more of Nestin, Musashi-1, SOX1, SOX2 and SOX3. In some instances, the hNPs express at least one of Nestin, Musashi-1, SOX1, SOX2 and SOX3, but do not express OCT4. In some other instances, the hNPs express at least two of Nestin, Musashi-1, SOX1, SOX2 and SOX3, but do not express OCT4. In yet another instance, the hNPs express at least three of Nestin, Musashi-1, SOX1, SOX2 and SOX3, but do not express OCT4. In a specific instance, the hNPs express SOX1, SOX2 and SOX3, but do not express OCT4. Neural progenitor cells may be cultured with or without feeder cells. In some instances, neural progenitor cells produced according to the methods presented in U.S. Pat. No. 7,531,354, are feeder cell free as well as free from embryoid bodies.

The disclosed EVs can be obtained in some instances by culturing differentiated neural cells, such as glial cells, derived directly or indirectly from pluripotent stem cells in cell culture medium under conditions and for a time sufficient to produce EVs, and isolating said EVs from the culture medium. Types of glial cells include oligodendrocytes, astrocytes, ependymal cells, Schwann cells, microglia, and satellite cells. In one instance, the differentiated neural cells (e.g., glial cells) comprise astrocytes. Differentiated neural cells that can be used include hN2™ neuronal cells (ARUNA BIOMEDICAL INC.), NeuroNet™ neurons, and AstroPro™ astrocytes (ARUNA BIOMEDICAL INC.).

EVs can be isolated from cell culture medium or tissue culture supernatant. EVs produced from cells can be collected from the culture medium by any suitable method. Typically, an isolated population of EVs can be prepared from cell culture or tissue supernatant by centrifugation, size exclusion columns, microfluidic devices, polymer precipitation, filtration or combinations of these methods. For example, the EVs can be prepared as described in U.S. Patent Application Document No. 20140356382, which is hereby incorporated by reference in its entirety. For example, EVs can be prepared by differential centrifugation, that is low speed (<2,0000 g) centrifugation to pellet larger particles followed by high speed (>100,000 g) centrifugation to pellet EVs, size filtration with appropriate filters (for example, 0.22 μm filter), gradient ultracentrifugation (for example, with sucrose gradient) or a combination of these methods.

In some embodiments, the EV-producing NP cells and/or neural cells disclosed herein are cultured for about 1, 2, 3, 4, 5, 6, 7, 8, 9, 10, 11, 12, 13 or 14 days or for as long as about 1, 2, 3, 4, 5, 6, 7, 8 weeks or about 1, 2, 3, 4, 5, or 6 months, depending on the cell and its ability to produce EVs. The EV-producing cells may be cultured in suitable media and grown under conditions that are readily determined by one of ordinary skill in the art. Cell culture conditions may vary with cell type and the examples presented hereinafter illustrate suitable media and conditions. For example, CMRL 1066 medium (from Invitrogen) with fetal bovine serum (e.g., at 10%) and optionally supplemented with glutamine or glutamine-containing mixtures and antibiotics could be used. Cells can be grown adhering on a surface in some embodiments, e.g., they can be grown as a monolayer to multilayers on the surface (feeder cell free) and may be grown until 30, 40, 50, 60, 70, 80, 90, 95 or 100% confluent. In other embodiments, the cells can be grown as cell aggregates in suspension cultures.

Cell growth media are well known in the art and comprise at least a minimum essential medium plus one or more optional components such as growth factors, ascorbic acid, glucose, non-essential amino acids, salts (including trace elements), glutamine, insulin (where indicated and not excluded), Activin A, transferrin, beta mercaptoethanol, and other agents well known in the art and as otherwise described herein. A preferred media is a low protein, serum-free based growth medium that supports neural cells. The growth factor used can be fibroblast growth factor 2(FGF 2 ), alone or preferably in combination with leukemia inhibitor factor (LIF). Depending on the NP or neural cells to be grown in the growth media, the inclusion of LIF is preferred but may not be required. Additional media includes basal cell media which may contain serum, for example, between about 0.1% and 20% (preferably, about 2-10%) fetal calf serum, or for defined medium, an absence of fetal calf serum and KSR, and optionally including bovine serum albumin (about 1-5%, preferably about 2%). In some instances, the medium is defined and is serum-free and has low protein content. In other instances, the media is media and supplement from Aruna which allow neural cultures to maintain a stable karyotype over multiple passages without the need for feeder cells, making them an excellent choice for a wide variety of research applications including early stage drug discovery. The components of the growth media depend on the type of neural cell to be grown, all of which are well known in the art. In one instance, a AB2™ Neural Cell Culture Media Kit is used and it contains AB2™ Basal Neural Medium and ANS™ Neural Medium Supplement. In a specific instance, the medium and supplement described in the instance above are specifically engineered for versatility to meet all neural cell culture needs. The AB2™ Basal Neural Medium and ANS™ Neural Medium Supplement can be used as the base for specialized mediums to direct differentiation of the hNP1™ line toward various neural phenotypes. Each lot of medium and supplement is pre-qualified for use by testing for cell growth, sterility, pH, osmolarity, and endotoxins.

Other agents which optionally may be added to the medium include, depending on the cell type grown in the media, for example, any one or more of nicotinamide, members of TGF-β family, including TGF-β 1, 2, and 3, Activin A, nodal, Bone Morphogen Proteins (BMP 2 to 7) serum albumin, members of the fibroblast growth factor (FGF) family, platelet-derived growth factor-AA, and-BB, platelet rich plasma, insulin growth factor (IGF-I, II, LR-IGF), growth differentiation factor (GDF-5,-6,-8,-10, 11), glucagon like peptide-I and II (GLP-I and II), GLP-1 and GLP-2 mimetobody, Exendin-4, parathyroid hormone, insulin, progesterone, aprotinin, hydrocortisone, ethanolamine, epidermal growth factor (EGF), gastrin I and II, copper chelators such as, for example, triethylene pentamine, forskolin, Na-Butyrate, betacellulin, ITS, noggin, neurite growth factor, nodal, valporic acid, trichostatin A, sodium butyrate, hepatocyte growth factor (HGF), sphingosine-1, VEGF, MG132 (EMD, CA), N2 and B27 supplements (Gibco, CA), steroid alkaloid such as, for example, cyclopamine (EMD, CA), keratinocyte growth factor (KGF), Dickkopf protein family, bovine pituitary extract, islet neogenesis-associated protein (INGAP), Indian hedgehog, sonic hedgehog, proteasome inhibitors, notch pathway inhibitors, sonic hedgehog inhibitors, heregulin, or combinations thereof, among a number of other components. Each of these components, when included, are included in effective amounts.

In some instances, suitable media may be made from the following components, such as, for example, Dulbecco's modified Eagle's medium (DMEM), Gibco #11965-092; Knockout Dulbecco's modified Eagle's medium (KO DMEM), Gibco #10829-018; Ham's F12/50% DMEM basal medium; 200 mM L-glutamine, Gibco #15039-027; non-essential amino acid solution, Gibco 11140-050; β-mercaptoethanol, Sigma #M7522; human recombinant basic fibroblast growth factor (bFGF), Gibco #13256-029.

Cell media are commercially available and can be supplemented with commercially available components, including defined xeno-free components, such as those available from Invitrogen Corp. (GIBCO), Cell Applications, Inc., Biological Industries, Beth HaEmek, Israel, and Calbiochem. One of ordinary skill in the art will be able to readily modify the cell media to produce any one or more of the target cells pursuant to the present disclosure.

The disclosed EV-producing cells may be cultured on a layer of feeder cells that support the cells in various ways. Approaches for culturing cells on a layer of feeder cells are well known in the art. The cells may be grown on a cellular support or matrix, as adherent monolayers, or cell aggregates in suspension. In some instances, the use of a cellular support may be preferred, depending upon the cells used to produce the EVs. When used, cellular supports preferably comprise at least one substrate protein. Substrate proteins include, for example, an extracellular matrix protein, which is a protein found in the extracellular matrix, such as laminin, tenascin, thrombospondin, and mixtures thereof, which exhibit growth promoting and contain domains with homology to epidermal growth factor (EGF) and exhibit growth promoting activity. Other substrate proteins which may be used include for example, collagen, fibronectin, vibronectin, polylysine, polyornithine and mixtures thereof. In addition, gels and other materials such as methylcellulose of other gels which contain effective concentrations of one or more of these embryonic stem cell differentiation proteins may also be used. Exemplary differentiation proteins or materials which include these differentiation proteins include, for example, recombinant laminin, BD Cell-Tak™ Cell and Tissue Adhesive, BD™ FIBROGEN Human Recombinant Collagen I, BD™ FIBROGEN Human Recombinant Collagen III, BD Matrigel™ Basement Membrane Matrix, BD Matrigel™ Basement Membrane Matrix High Concentration (HC), BD™ PuraMatrix™ Peptide Hydrogel, Collagen I, Collagen I High Concentration (HC), Collagen II (Bovine), Collagen III, Collagen IV, Collagen V, and Collagen VI, among others.

Alternatively, these cells may be cultured in a culture system that is free of feeder cells, or essentially free of feeder cells, but nonetheless supports proliferation of the cells to produce EVs. The growth of cells in feeder-free culture can be supported using a medium conditioned by culturing previously with another cell type. Alternatively, the growth of EV-producing cells in feeder-free culture without differentiation can be supported using a chemically defined medium. These approaches are well known in the art. In certain embodiments, the cells are grown in feeder cell free medium.

EVs can be harvested at various time intervals (e.g., at about 1, 2, 4, 6, 8 or 3, 6, 9, 12 day or longer intervals, depending upon the rate of production of EVs). Example yields of EVs can range from at least about 1 ng EVs/1 million cells to at least about 10.0 μg EVs/1 million cells (e.g., about 10 ng EVs/1 million cells, about 50 ng EVs/1 million cells, about 100 ng EVs/1 million cells, about 500 ng EVs/1 million cells, about 750 ng EVs/1 million cells, about 800 ng EVs/1 million cells, about 900 ng EVs/1 million cells, about 1.0 μg EVs/1 million cells, about 1.5 μg EVs/1 million cells, about 2.0 μg EVs/1 million cells, about 2.5 μg EVs/1 million cells, e.g. about 3.0 μg EVs/1 million cells, about 5.0 μg EVs/1 million cells) during a time period of about 24 hours to seven days of culture of proliferative and non-proliferative neural cells as otherwise described herein.

In many instances, EVs are harvested and collected by ultracentrifugation or differential centrifugation or any combination thereof, pelleted EVs are collected, and, optionally, collected pelleted EVs are washed with a suitable medium. For example, a preparation of EVs can be prepared from cell culture or tissue supernatant by centrifugation, filtration or combinations of these methods. In some embodiments, the EVs can be prepared by differential centrifugation, that is low speed (<2,0000 g) centrifugation to pellet larger particles followed by high speed (>100,000 g) centrifugation to pellet EVs, size filtration with appropriate filters (for example, 0.22 μm filter), gradient ultracentrifugation (for example, with sucrose gradient) or a combination of these methods. EVs may be purified by differential centrifugation, micro and ultra-filtration, polymeric precipitation, microfluidic separation, immunocapture and size-exclusion chromatography. These and/or related methods for isolating and purifying EVs are described by Théry et al. (Current Protocols in Cell Biology 3.22.1-3.22.29 (2006), copyright 2006 by John Wiley & Sons, Inc.), Sokolova et al. (Colloids and Surfaces B Biointerfaces 87:146-150 (2011)), Wiklander et al. (J Extracellular Vesicles 4:26316 (2015)), and Böing et al. (J Extracellular Vesicles 3:23430 (2014)). Other methods for isolation may be developed, such as electrical field radiofrequency and acoustics.

Methods of manufacturing synthetic vesicles, such as synthetic exosomes, are known in the art. Such methods may be used to produce synthetic vesicles suitable for use in the compositions and methods provided herein. It is noted that the contents of EVs, i.e., EVs in which the lipid bilayer has been removed or eliminated and the contents obtained, may also be used to engineer artificial EVs.

### (iii) Cargo to be Delivered to the CNS

In some aspects, provided herein are EVs comprising cargo, e.g., one or more therapeutic agents, for delivery to the CNS, e.g., across an intact blood brain barrier. In some embodiments, EVs are derived from neural cells (e.g., neural progenitor cells or neural stem cells). In some embodiments, EVs are derived from non-transformed human neural progenitor cells. In some embodiments, the cargo is exogenous cargo, which can be introduced into EVs by recombinant expression of the cargo in cells from which the EVs are derived, or introduced into EVs after the EVs are isolated from cells. In some embodiments, the exogenous cargo can include one more therapeutic agents. Virtually any molecular agent which may be used for the therapeutic management or treatment of a disease and/or disorder can be loaded into EVs. Therapeutic agents suitable for delivery via EVs encompass a wide variety of active agents, including (i) small molecule therapeutic agents synthesized via chemical synthesis, (ii) naturally derived compounds which may, e.g., be obtained via purification from natural sources, (iii) nucleic acid-based compounds of various kinds, e.g., oligonucleotides (such as siRNA, splice-switching RNA, CRISPR guide strands, short hairpin RNA (shRNA), antisense oligonucleotides, antisense RNA, micro RNA (miRNA)), polynucleotides (such as mRNA, plasmid, cDNA, DNA), and nucleic acids which are chemically synthesized and/or which comprise chemically modified nucleotides (such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc.), and (iv) peptides and polypeptides (i.e., proteins or antibodies) of any kind, including those obtained via peptide synthesis or via recombinant protein production.

Therapeutic agents can be obtained from essentially the entire space of pharmaceutically and/or pharmacologically and/or diagnostically relevant agents, for instance anti-inflammatory agents, anti-fibrotics, anticancer agents, cytostatic agents, tyrosine kinase inhibitors, statins, NSAIDs, antibiotics, antifungal agents, antibacterial agents, anti-inflammatory agents, anti-fibrotics, antihypertensives, aromatase or esterase inhibitors, an anticholinergics, SSRIs, BKT inhibitors, PPAR agonists, HER inhibitors, AKT inhibitors, BCR-ABL inhibitors, signal transduction inhibitors, angiogenesis inhibitors, synthase inhibitors, ALK inhibitors, BRAF inhibitors, MEK inhibitors, PI3K inhibitors, neprilysin inhibitors, beta2-agonists, CRTH2 antagonists, FXR agonists, BACE inhibitors, sphingosine-1-phosphate receptor modulators, MAPK inhibitors, Hedgehog signaling inhibitors, MDM2 antagonists, LSD1 inhibitors, lactamase inhibitors, TLR agonists, TLR antagonists, IDO inhibitors, ERK inhibitors, Chk1 inhibitors, splicing modulatory, DNA or RNA intercalators, etc. Other non-limiting examples of pharmacological agents as per the present invention includes for instance everolimus, trabectedin, abraxane, pazopanib, enzastaurin, vandetanib, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase (mek) inhibitor, pemetrexed, erlotinib, dasatanib, nilotinib, decatanib, panitumumab, amrubicin, oregovomab, nolatrexed, batabulin, ofatumumab, zanolimumab, edotecarin, tetrandrine, rubitecan, tesmilifene, oblimersen, ticilimumab, ipilimumab, gossypol, cilengitide, gimatecan, lucanthone, neuradiab, vitespan, talampanel, atrasentan, romidepsin, sunitinib, 5-fluorouracil, vorinostat, etoposide, gemcitabine, doxorubicin, 5′-deoxy-5-fluorouridine, vincristine, temozolomide, seliciclib, capecitabine, camptothecin, PEG-labeled irinotecan, tamoxifen, toremifene citrate, anastrazole, exemestane, letrozole, vatalanib, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, erlotinib, lapatanib, canertinib, lonafarnib, tipifarnib, amifostine, suberoyl analide hydroxamic acid, valproic acid, trichostatin sorafenib, arnsacrine, anagrelide, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, squalamine, endostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, droloxifene, 4-hydroxytamoxifen, pipendoxifene, arzoxifene, fulvestrant, acolbifene, lasofoxifene, idoxifene, topotecan, rapamycin, temsirolimus, zolendronate, prednisone, lenalidomide, gemtuzumab, hydrocortisone, dexrazoxane, alemtuzumab, all-transretinoic acid, ketoconazole, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, efavirinz among others. Furthermore, therapeutic agents also include naturally-derived compounds which may for instance be obtained via purification from natural sources, any type of nucleic acid-based compounds, for instance oligonucleotides such as siRNA, splice-switching RNA, CRISPR guide strands, short hairpin RNA, antisense oligonucleotides, mRNA, and in particular nucleic acid-based agents which are chemically synthesized and/or which comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc. Furthermore, peptides and polypeptides, and not only peptides and/or proteins obtainable via peptide synthesis but also peptides and proteins obtainable through recombinant protein production, are also contemplated as therapeutic agents suitable for use in the compositions and methods of the present disclosure.

In some embodiments, the EVs comprise one or more inhibitory nucleic acids. For example, in some embodiments, the EVs can comprise one or more inhibitory nucleic acids selected from short interfering RNAs (siRNAs), short hairpin RNAs (shRNA), micro RNAs (miRNAs), antisense oligonucleotides (ASOs), and double-strand RNAs (dsRNA).

In some embodiments, the EVs comprise one or more therapeutic antibody or antigen-binding portion thereof, including an antibody fragment selected from the group consisting of a Fab, a F(ab′)₂, an scFv, a tandem scFv, a diabody, a minibody, and a single domain antibody. In some embodiments, the antibody, or antigen binding portion thereof, is a humanized antibody, or antigen binding portion thereof. In some embodiments, the antibody, or antigen binding portion thereof, is a fully human antibody, or antigen binding portion thereof. In some embodiments, the EVs comprise one more neurotrophic agents. The EVs can comprise one or more agents selected from leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4(BMP4 ), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

The foregoing therapeutic agents and other cargo can be loaded into EVs using art-recognized methods. Such methods include, but are not limited to, lipofection, transfection, electroporation, click chemistry, conjugation to a hydrophobic moiety, or recombinant expression (for peptide or protein based therapeutics) of the therapeutic agent as a fusion protein with an exosomal sorting domain, such as the exosomal sorting domain of CD9, CD63, CD81, or Lamp2b, or any of the additional exosomal sorting domains described herein. Peptide or protein therapeutic agents can also be recombinantly expressed as a fusion protein with an exosomal protein (or an exosomal sorting domain thereof) typically found in the EV lumen, such as ALIX or syntenin. In other embodiments, the therapeutic agent can be loaded into EVs by coupling the agent to a cell penetrating peptide, such as, for example, transportan, penetratin, CADY-1,or VP22. Additional cell penetrating peptides are described, for example, in US20109/0388347A1, the entire contents of which are incorporated herein by reference.

Any of the foregoing agents, or combinations thereof, can be loaded into EVs as described herein, for delivery to the brain and/or CNS, e.g., across the blood-brain barrier.

### III. Inhibiting Necroptosis and/or Reducing Inflammation

A method of inhibiting necroptosis and/or reducing inflammation in a cell is provided. The method includes contacting the cell with a therapeutically effective amount of a composition comprising extracellular vesicles (EVs), e.g., neural EVs, neural progenitor EVs, thereby inhibiting necroptosis and/or reducing inflammation.

“Necroptosis” as used herein refers to a programmed necrotic cell death pathway. Receptor interacting protein kinase 1 (RIPK1) regulates necroptosis, by promoting the sequential activation of two downstream targets, receptor interacting protein kinase 3(RIPK3 ) and mixed lineage kinase domain like pseudokinase (MLKL). Application of Nec-1s, a highly specific inhibitor of RIPK1 kinase activity, blocks necroptosis and inflammation in vitro and in vivo (Ito et al. 2016 Science 353(6299): 603-608). Without wishing to be bound by theory, necroptosis may play a role in the pathogenesis of neurodegenerative disease, including ALS, while the confirmation requires further preclinical and clinical studies. Some studies detected robust activation of RIPK1, RIPK3, and MLKL proteins in preclinical as well as clinical studies of ALS, while other studies did not show involvement of necroptotic pathway in ALS. The studies showing the activation of the necroptotic pathway in ALS are based on consistent results assessing all molecular steps of this cascade namely RIPK1, p-RIPK1, RIPK3, MLKL, p-MLKL, or p-RIPK3, and tumor necrosis factor (TNF)-α, including in situ experiments focusing selectively on the cells of upmost interest, i.e., motor neurons of the anterior horn of the spinal cord.

In some embodiments, level of p-RIPK1 is decreased in the cell contacted with the therapeutically effective amount of the composition comprising EVs relative to a control cell.

In some embodiments, the cell is within a subject. In some embodiments, the subject has amyotrophic lateral sclerosis (ALS), and wherein neurological performance, motor performance, and/or one or more signs or symptoms associated with ALS are improved, and/or survival is prolonged in the subject relative to a control subject. The control subject can be a subject that does not contain the cell contacted with the therapeutically effective amount of the composition comprising the EVs.

In some embodiments, inflammation is reduced in the cell contacted with the therapeutically effective amount of the composition comprising the EVs relative to a control cell. A control cell can be a cell not contacted with the therapeutically effective amount of the composition comprising the EVs. For example, the number of regulatory T cells is increased, the number of T helper cells is reduced, the number of M2 macrophages is increased, an inflammatory response of M1 macrophages is suppressed, an anti-inflammatory response of M2 macrophages is increased, the level of pro-inflammatory cytokines (e.g., IL-17) is reduced, and/or the level of anti-inflammatory cytokines (e.g., IL-10) is increased in the cell contacted with the EV composition relative to a control cell.

### IV. Formulation, Delivery, and Administration

The EVs provided herein can be formulated in a pharmaceutical composition for delivery to a subject. In some embodiments of the present disclosure, subjects are in need of delivery of EVs to the CNS. In some embodiments, subjects are in need of delivery of cargo loaded in EVs to the CNS. In some embodiments, subjects are in need of reducing swelling in the brain, reducing inflammation in the brain, or reducing the volume of a lesion in the brain. In some embodiments, subjects have a neurological condition or a symptom associated with the neurological condition.

Pharmaceutical compositions can comprise a therapeutically effective amount of the present EVs (e.g., comprising neural cell derived EVs) and a pharmaceutically acceptable carrier. Formulations containing the disclosed EVs may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions typically include a conventional pharmaceutical carrier and/or excipient and may additionally include other medicinal agents, carriers, adjuvants, additives and the like. The weight percentage ratio of the EV s to the one or more excipients can he between about 20:1 to about 1:60, or between about 15:1 to about 1:45, or between about 10:1 to about 1; 40, or between about 9:1, 8:1, 7:1, 6:1, 5:1, 4:1, 3:1, 2:1, or 1:1 to about 1:2, 1:3, 1:4, 1:5, 1:6, 1:7, 1:8, 1:9, 1:10, 1:15, 1:20, 1:25, 1:30, or 1:35, and preferably is about 20:1, 19:1, 18:1, 17:1, 16:1, 15:1, 14:1, 13:1, 12:1, 11:1, 10:1, 9:1, 8:1, 7:1, 6:1, or 5:1. In some embodiments, the disclosed composition comprises between about 1 μg to about 1 g or more of total EVs, about 500 μg about 500 mg, about 1 mg to about 500 mg of total EVs, about 5 mg to about 500 mg, about 10 mg to about 500 mg, about 25 mg to about 500 mg, about 50 mg to about 350 mg, about 75 mg to about 450 mg, about 50 mg to about 450 mg, or about 75 mg to about 325 mg, or about 100 mg to about 650 mg of total EVs and may optionally contain one or more suitable pharmaceutical carriers, additives and/or excipients.

An injectable composition for parenteral administration (e.g., intravenous, intramuscular, intranasal), will typically contain the EVs and optionally additional components in a suitable i.v. solution, such as sterile physiological salt solution. The composition may also be formulated as a suspension in an aqueous emulsion.

Liquid compositions can be prepared by dissolving or dispersing the pharmaceutical composition comprising the EVs, and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline. In the case of intranasal or intrapulmonary administration, the compositions may be provided as liquid composition which can be sprayed into the nose, trachea and/or lungs.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

In some embodiments, the disclosed EVs are contained in or on a biocompatible scaffold, such as a hydrogel. In some embodiments, the disclosed EVs are contained in or on a biocompatible scaffold comprising collagen, fibrin, silk, agarose, alginate, hyaluronan, chitosan, a biodegradable polyester such as polylactic-co-glycolic acid, polylactic acid, or polyglycolic acid, polyethylene glycol, polyvinylpyrrolidone, polyethersulfone, a peptide-based biomaterial, glycose amino glycan, fibronectin, laminin, or any combination thereof.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or are apparent to those skilled in the art; for example, see Remington's Pharmaceutical Sciences (17th Ed., Mack Pub. Co. 1985). The composition to be administered will contain a quantity of the selected compound in a pharmaceutically effective amount for therapeutic use in a biological system, including a patient or subject according to the present invention.

Intravenous formulations can comprise the EVs described herein, an isotonic medium and one or more substances preventing aggregation of the EVs. Example intravenous fluid formulations may contain saline solutions (e.g., normal saline (NS); about 0.9% w/v of NaCl, about 300 mOsm/L) and/or dextrose 4% in 0.18% saline, and optionally 1%, 2%, or 3% human serum albumin. In addition, the EVs may be disrupted to obtain the contents and the contents used in compositions according to the present invention.

In exemplary embodiments, formulations of the invention may comprise about 50 ng EVs/ml intravenous medium, including about 100 ng-1.0 μg, 1.0 μg-100 μg, or more EVs/ml intravenous medium.

In some embodiments, intravenous formulations may comprise about 0.1-20.0 μg EVs/ml intravenous medium, or about 20.0 μg or more EVs/ml intravenous medium.

In some embodiments, the pharmaceutical composition is in a dosage form comprising at least 25 mg of EVs, at least 300 mg of EVs, about 350 mg-1 g of EVs, or about 1 g (1,000 mg) or more of EVs, alone or in combination with a therapeutically effective amount of at least one additional bioactive agent, which agent may be useful in the treatment of neurological disorders and injuries, including ALS. In some embodiments, the pharmaceutical composition comprises between about 10 mg to about 750 mg, about 25 mg to about 650 mg, or between about 30 mg to about 500 mg, or about 35 mg to about 450 mg, most often about 50 to about 500 mg of EVs.

Compositions comprising the EVs provided herein can administered to a subject at a site distal to the CNS by any suitable route, including but not limited to, auricular (otic), buccal, conjunctival, cutaneous, dental, electro-osmosis, endocervical, endosinusial, endotracheal, enteral, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracavernous, intracavitary, intracorneal, intracoronal (dental), intracoronary, intracorporus cavernosum, intradermal, intraductal, intraduodenal, intraepidermal, intraesophageal, intragastric, intragingival, intraileal, intralesional, intraluminal, intralymphatic, intramuscular, intraocular, intraovarian, intrapericardial, intraperitoneal, intrapleural, intraprostatic, intrapulmonary, intrasinal, intrasynovial, intratendinous, intratesticular, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, parenteral, percutaneous, periarticular, perineural (peripheral), periodontal, rectal, respiratory (inhalation), retrobulbar, soft tissue, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease, signs, or symptoms to be treated. In some embodiments, the EV composition is administered directly to the CNS, e.g., by intrathecal administration or direct injection to the brain.

Administration of EVs as described herein is suitable for delivering the EVs to the brain or CNS, and in some embodiments, across the blood-brain barrier. In some embodiments, administration is at the site of diseased and/or dysfunctional tissue (e.g., brain). In some embodiments, the site of administration is distal to the site of diseased and/or dysfunctional tissue (e.g., in the case of intravenous or intranasal delivery). In one embodiment, the EV composition described herein is administered to a subject parenterally. In one embodiment, the EV composition described herein is administered to a subject intravenously. In another embodiment, the EV composition described herein is administered to a subject intranasally. In some embodiments, the subject has an intact or largely intact blood-brain barrier.

An injectable composition for parenteral administration (e.g. intravenous, intramuscular, or intranasal), can contain the EVs of the present disclosure and optionally additional components in a suitable i.v. solution, such as sterile physiological salt solution. In other embodiments, the composition is formulated as a suspension in an aqueous emulsion.

Pharmaceutical compositions comprising the EVs as described herein may be administered to a subject as a monotherapy (a single agent) or in a combination therapy where the subject is administered a pharmaceutical composition comprising the EVs in combination with one or more additional agents. A pharmaceutical composition comprising the EVs of the present disclosure, and one or more additional agents, can be administrated to a subject simultaneously, sequentially or temporally.

A therapeutically effective amount of a pharmaceutical composition comprising the EVs of the present disclosure, is an amount of sufficient to treat or ameliorate one or more symptoms of the condition being treated (e.g., a neurological disorder, for example, Alzheimer's Disease, Huntington's Disease, Parkinson's Disease, or neurological cancer, e.g., glioblastoma), while not exceeding an amount which may cause significant adverse effects. Dosages that are therapeutically effective can depend on many factors including the nature of the condition to be treated as well as the particular individual being treated.

In certain aspects, EVs, e.g., neural EVs, neural progenitor EVs, EVs derived from non-transformed neural progenitor cells, of the present disclosure, or pharmaceutical compositions comprising EVs of the present disclosure have superior ability to cross the blood brain barrier and are more efficiently delivered to the CNS of subjects compared to reference molecules, e.g., blood-derived exosomes, when administered at a site distal to the CNS, achieving a concentration in the CNS, a concentration in the brain, a brain-blood ratio, or a plasma-blood ratio that is at least 10% greater, 15%-1000% greater, or more than 1000% greater than a concentration in the CNS, a concentration in the brain, a brain-blood ratio, or a plasma-blood ratio of molecules being compared, e.g., blood-derived exosomes, non-neural EVs.

In some aspects, delivery to the CNS of the EVs of the present invention, or pharmaceutical composition comprising the EVs of the present invention, causes reduction of necroptosis or inflammation in the CNS. In some aspects, delivery to the CNS of the EVs of the present invention, or pharmaceutical composition comprising the EVs of the present invention, causes improvement in a neurological condition or a symptom associated with the ALS (e.g., muscle weakness, walking difficulty, tripping, falling, hand weakness, hand clumsiness, slurred speech, chewing difficulty, swallowing difficulty, and/or breathing difficulty) in a subject. In some aspects, delivery to the CNS of the EVs of the present invention, or pharmaceutical composition comprising the EVs of the present invention, causes improvement of survival (e.g., prolonged survival) of the subject. In some aspects, any one of these effects (e.g., reduction of necroptosis or inflammation in the CNS, improvement in a neurological condition or a symptom associated with ALS, prolonged survival) in a subject caused by the EVs of the present invention, or pharmaceutical compositions comprising EVs of the present invention, is at least 10% greater, 15%-1000% greater, or more than 1000% greater than the effect caused by molecules being compared, e.g., vehicle, blood-derived exosomes, non-neural EVs, or cargo administered without EVs.

### V. Treating Amyotrophic Lateral Sclerosis (ALS)

In some aspects, the disclosure provides a method of treating a subject having amyotrophic lateral sclerosis (ALS) using the EVs or compositions comprising the EVs described herein. Any of the compositions or pharmaceutical compositions containing the EVs described herein are suitable for use in any of the methods provided herein. In some embodiments, the EVs are derived from neural cells, e.g., neural progenitor cells, neurons, or astrocytes. In other exemplary embodiments, the EVs are produced synthetically, and contain one or more markers characteristic of neural EVs, e.g., one or more proteins or nucleic acids present in neural EVs that are absent in EVs derived from mesenchymal stem cells. In other embodiments, the EVs contain one or more additional therapeutic agents, for example, one or more of riluzole, edaravone, or sodium phenylbutyrate-taurursodiol, or one or more medications to alleviate or control one or more signs or symptoms including muscle cramps, constipation, fatigue, excessive oropharyngeal secretion, pain, depression, sleep difficulty, mood disturbance, urinary incontinence, or leg swelling.

In some aspects, the invention provides a method of treating (e.g., curing, suppressing, ameliorating associated symptoms of, delaying or preventing the progression of, delaying or preventing onset of, or preventing recurrence or relapse of) amyotrophic lateral sclerosis (ALS) in a subject, by administering to the subject a composition containing EVs as described herein, in an amount sufficient to treat ALS in the subject. The amount sufficient to treat the disease or disorder (e.g., ALS) is preferably an effective amount, e.g., a therapeutically effective amount, as provided herein.

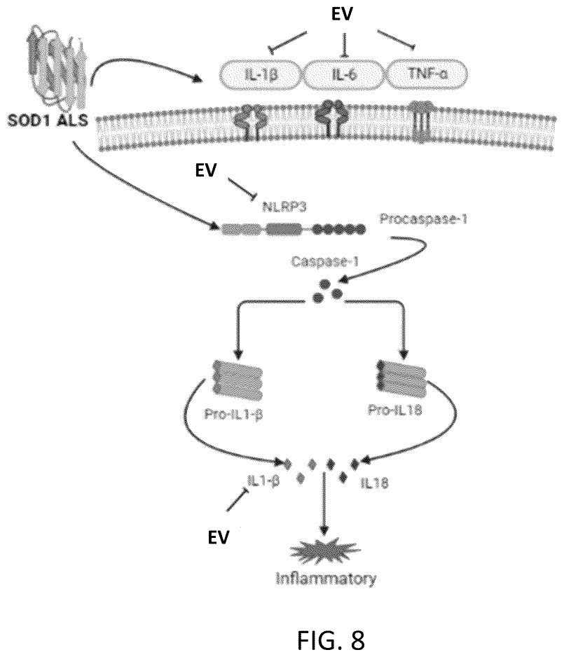

As shown in FIG. 8, the EVs (e.g., neural EVs, neural progenitor derived EVs) provided herein can treat ALS via several underlying mechanisms of action. Without wishing to be bound by theory, the EVs provided herein can inhibit pro-inflammatory cytokines (e.g., IL-1β, IL-6, TNFα), the NLRP3 pathway (e.g., NLRP3, caspase-1, IL1-β, IL-18), and/or necroptosis in the cell or the subject.

The EV composition can be administered by any suitable formulation, route, and dosage to achieve the desired therapeutic effect. For example, the EV composition can be administered to a site distal to the central nervous system (CNS) of the subject. In some embodiments, the composition is administered intravenously, intranasally, intraperitoneally, orally, or by combination of any thereof. Alternatively or additionally, the EV composition can be administered directly to the CNS, such as by injection to the brain or intrathecal administration.

In some embodiments, by the methods provided herein, neurological performance, motor performance, and/or one or more signs or symptoms associated with ALS are improved, and/or survival is prolonged in the subject relative to a control subject. In some embodiments, the one or more signs or symptoms include muscle weakness, walking difficulty, tripping, falling, hand weakness, hand clumsiness, slurred speech, chewing difficulty, swallowing difficulty, and/or breathing difficulty.

In some embodiments, by the methods provided herein, level or activity of neurofilament light chain (NFL), chitinase-3-like protein 1(CHI3L1 ), nucleotide-binding domain leucine-rich repeat containing family pyrin domain containing 3 (NLRP3), a NLRP3 signaling pathway molecule, and/or a proinflammatory cytokine is decreased in the subject. Without wishing to be bound by theory, NFL is the main byproducts of neuroaxonal breakdown, and the NFL levels can indicate neurodegeneration activity in ALS. CHI3L1 is a marker of glial activation, and the CHI3L1 levels can indicate neuroinflammatory status, disease severity, and progression in ALS.

In some embodiments, the NLRP3 signaling pathway molecule includes caspase-1, IL1-β, and/or IL-18. In some embodiments, the proinflammatory cytokine comprises TNFα and/or IL1-β.

The activities of EV s described herein can be evaluated by methods known in the art. The amount of EVs required for use in treatment can vary not only with the particular cell from which the EVs are prepared, but also with the route of administration, the nature of the condition being treated and the age and condition of the patient and can be ultimately at the discretion of the attendant physician or clinician. In general, however, a dose can be in the range of from about 0.01 mg/kg to about 10 mg/kg of body weight per day.

Alteration of symptoms as a result of treatment can be measured relative to any suitable control. For example, alteration of symptoms can be measured relative to the frequency, severity, or duration, or number of symptoms experienced by the same subject prior to initiating treatment. In other embodiments, alteration of symptoms can be measured relative to the frequency, severity, duration, or number of symptoms experienced by a different subject, or group of subjects, with like symptoms who do not receive the treatment, e.g., who do not receive a composition containing EVs. In some embodiments, the degree of improvement is at least 5%, as determined relative to a suitable control.

In some embodiments, a composition containing EVs is administered to a subject as a single dose. In some embodiments, a composition containing EVs is administered in multiple doses. For example, the composition can be administered, in some embodiments, once every day, once every 2 days, once every 3 days, once every 4 days, once every 5 days, once every 6 days, once every 7 days, once every 2 weeks, once every 3 weeks, once every 4 weeks, once every 8 weeks, or once every 12 weeks.

In some embodiments, the EV composition of the present disclosure, is administered in combination with a second therapy known to be effective in treating ALS or preventing, alleviating, or treating one or more signs, symptoms, conditions, or complications associated with ALS. The EV composition may be administered before, after, concurrent with, or in conjunction with, the second therapy.

The second therapy may be an additional therapeutic agent for ALS, such as riluzole, edaravone, or sodium phenylbutyrate-taurursodiol, or medication to alleviate or control one or more signs or symptoms including muscle cramps, constipation, fatigue, excessive oropharyngeal secretion, pain, depression, sleep difficulty, mood disturbance, urinary incontinence, and leg swelling. The EV composition and the additional therapeutic agent can be administered in combination within the same composition or co-administered as separate compositions. The second therapy can also be non-agent therapy, such as respiratory therapy, physical therapy, occupational therapy, speech therapy, nutritional therapy, and psychosocial therapy.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting.

### EXAMPLES

### Example 1. Isolation of Extracellular Vesicles (EVs)

The following example describes isolation of extracellular vesicles (EVs) for use in the compositions and methods described herein.

A purified population of EVs was obtained from neural progenitor cells in accordance with the methods provided in U.S. Patent Application Publication No. US2018/0327714A1, which is incorporated herein by reference in its entirety. Briefly, human neural progenitor cells were cultured, and medium was collected from confluent cultures 24 hours post media change. Medium was filtered through a 0.22 μm filter unit, and frozen at −20° C. Prior to purification of EVs, the frozen medium was thawed overnight at 4° C. EVs were purified from filtered cell culture medium using tangential flow filtration (TFF) and chromatography.

### Example 2. Neural EVs Downregulates a Marker of Necroptosis in Vitro

This example describes the effect of neural EVs to inhibit necroptosis and inflammation in vitro.

The human microglial cell line (HMC3) were pretreated with vehicle or the neural progenitor EVs (neural EVs) prepared in Example 1 (middle and high dose) for 2 h before TNF-α (10ng/mL) stimulation for 4 h and measured for p-RIPK1 by western blot. Twenty-four (24) hours prior to treatment, 300,000 HMC3 cells (ATCC, CRL-3304) per well were plated in 6-well dishes. Cells were treated with TNF-alpha (10 ng/mL, R&D Systems) for 4 hours in the presence of either vehicle or neural EVs (middle dose: 5×10⁴ EVs/cell; high dose: 1×10⁵ EVs/cell). After 4 hours, cells were lysed, and protein quantified using a BCA assay. Cell lysates were run on a gel and stained for phospho-RIPK1 (ProteinTech, #28252-1-AP) and GAPDH (GeneTex, #GTX100118). Gels were quantified with ImageJ. Phospho-RIPK1 can be an indicator of the extent of necroptosis in the cells (Chevin & Sébire 2021 Cell Death Discovery 7:79).

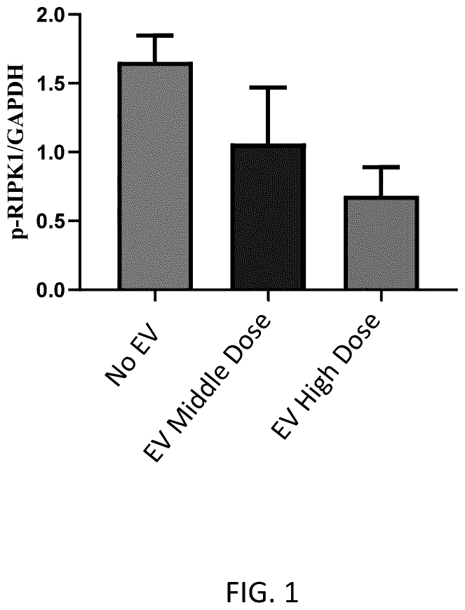

As shown in FIG. 1, the immunoblot quantification, as normalized to GAPDH (phospho-RIPK1/GAPDH ratios) were decreased in cells pretreated with EVs in a dose-dependent manner, indicating the effect of EVs to down-regulate TNF-α induced p-RIPK1 upregulation and necroptosis.

### Example 3. Neural EVs Delay Disease Progression and Prolongs Survival in ALS Mouse Model

This example describes the effect of neural EVs to delay disease progression, ameliorate signs and symptoms, and improve survival of ALS mice.

B6SJL-Tg(SOD1*G93A)1Gur/J mice were used to test the effect of the neural EVs on ALS progression. This transgenic model, which expresses large amounts of mutant superoxide dismutase 1 (SOD1), develops neurodegeneration of spinal motor neurons, leading to progressive paralysis similar to ALS.

Beginning on day 62 of age, 40 B6SJL-Tg(SOD1*G93A)1Gur/J male mice received either a weekly intravenous (IV) dose of the neural progenitor EVs (neural EVs) prepared in Example 1 (2.7×10¹¹ particles/kg) or vehicle from weeks 1-3 (n=20 per group), and either a weekly intranasal (IN) dose of the neural EVs (2×10⁹ total particles) or vehicle from weeks 4-9 (up to 9 administrations total). Neurological deficit score (NDS) scoring and rotarod testing were used to quantify disease progression. Beginning on Day 59, mice were weighed and NDS was measured twice weekly. Beginning on Day 90, mice were weighed and NDS was checked daily until the humane endpoint. To quantify motor function decline, mice underwent rotarod testing weekly beginning the same week as NDS. Evaluations continued on this schedule until mice reached their humane endpoint or day 125-126, whichever came first. Mice were then euthanized and samples were collected for additional analysis.

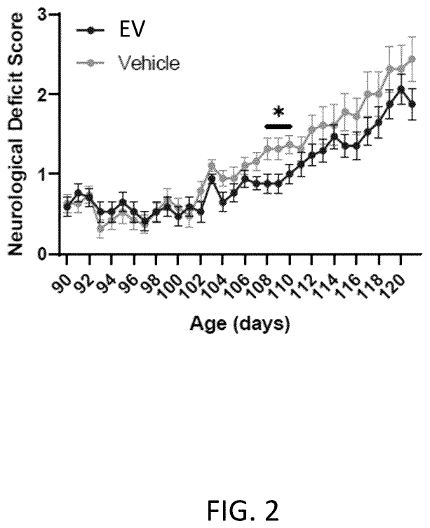

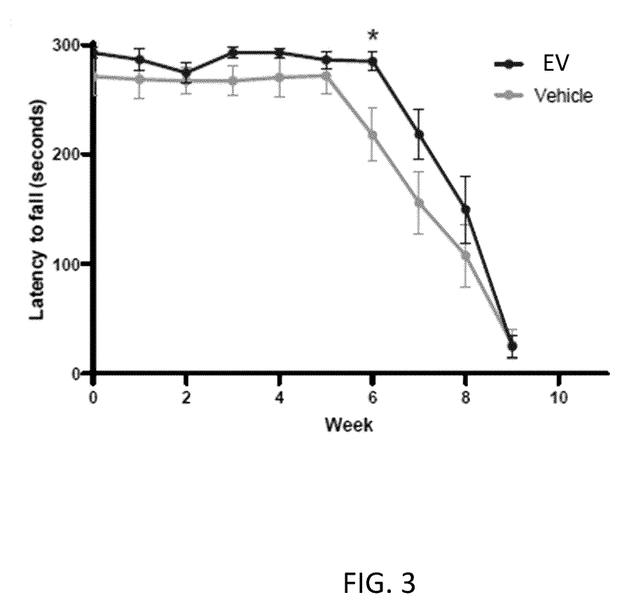

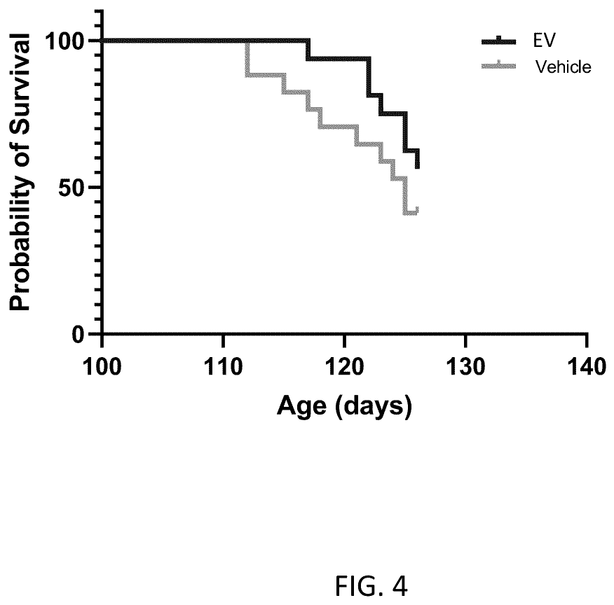

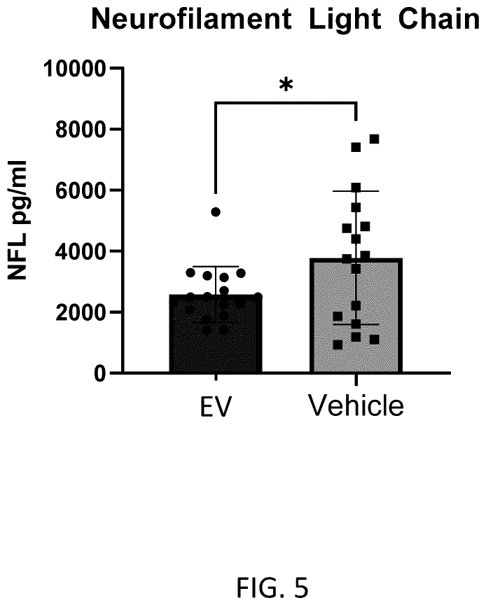

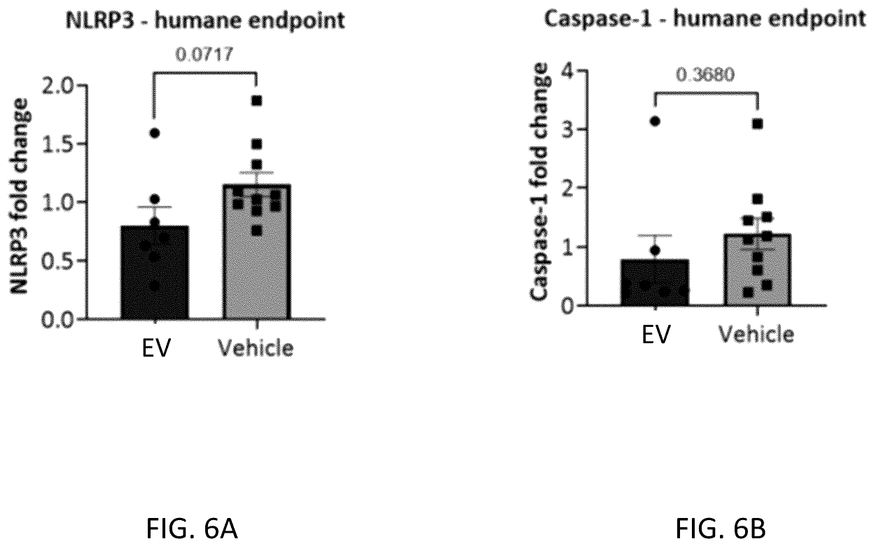

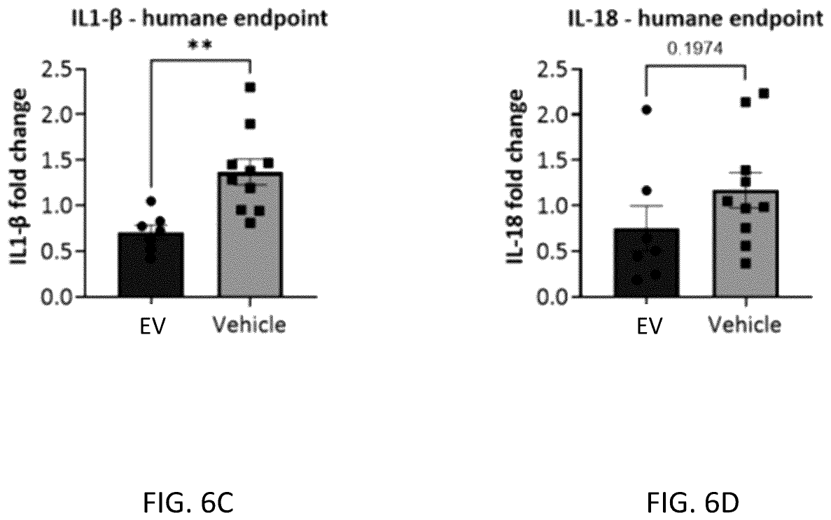

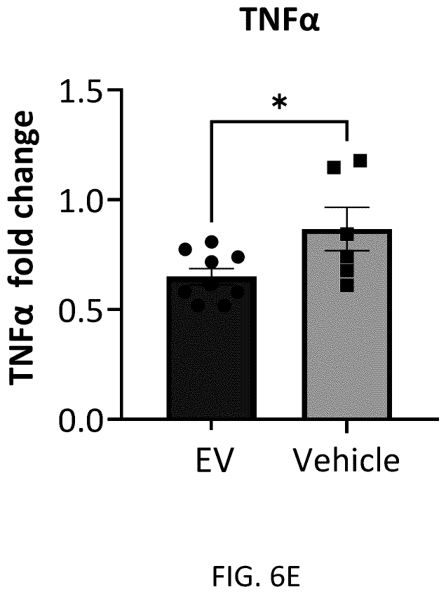

As shown in FIGS. 2-4, treatment with neural EVs improved NDS score (FIG. 2) and rotarod performance (FIG. 3), as well as prolonged survival (FIG. 4), demonstrating the effect of the neural EVs to delay disease progression. Further, treatment with neural EVs showed a significant decrease in neurofilament light chain levels in the serum at Day 97 (FIG. 5), indicating decreased neurodegeneration activity. On the transcript level in the lumbar spinal cord, treatment with the neural EVs significantly reduced proinflammatory cytokines tumor necrosis factor alpha (TNFα, FIG. 6E) and Interleukin-1 beta (IL1-β, FIG. 6C). The neural EV treatment also led to a trending decrease in several targets related to the NLRP3 inflammatory pathway, including NLRP3 (FIG. 6A), Caspase 1 (FIG. 6B), IL1-β (FIG. 6C), and IL-18 (FIG. 6D).

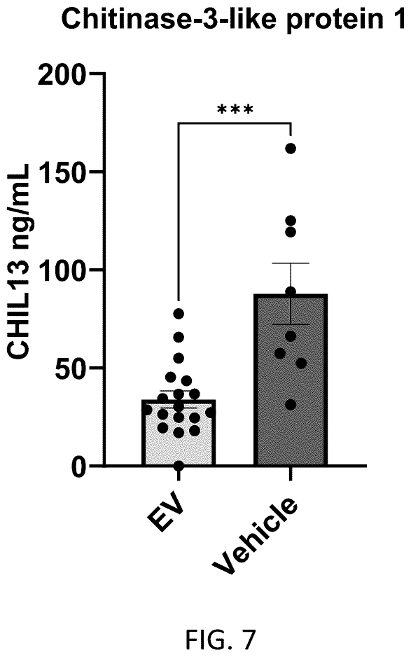

In a separate experiment, beginning on day 69 of age, 30 B6SJL-Tg (SOD1*G93A)1Gur/J male mice received intranasal (IN) administrations of either the neural progenitor EVs (neural EVs) prepared in Example 1 (4.2×10¹¹ particles/kg, 1.1×10¹⁰ total particles; n=20) or vehicle (EV formulation buffer, n=10) five times weekly (i.e., Monday-Friday) until a humane endpoint was reached. Serum samples were collected on Day 80 to quantify protein levels of chitinase- 3-like protein 1(CHI3L 1 ), a marker of glial activation and ALS disease progression, by ELISA. As shown in FIG. 7, the neural EV-treated mice had significantly (p=0.0002) lower levels of CHI3L1 compared to vehicle treated mice, indicating that intranasally administered neural EVs delay glial cell activation and disease progression in an ALS mouse model.

In summary, these results indicate that the neural EVs reduce inflammation (e.g., the NLRP3 inflammasome signaling), delay disease progression, delay motor function impairment, and increase survival time in vivo in the mouse model of ALS.

### INCORPORATION BY REFERENCE

The contents of all references, patents, pending patent applications, and publications cited throughout this application are hereby expressly incorporated by reference herein in their entirety.

### EQUIVALENTS

Those skilled in the art will recognize or be able to ascertain using no more than routine experimentation, many equivalents to the specific embodiments of the invention described herein. Such equivalents are intended to be encompassed by the following claims.

What is claimed is:

1. A method of treating amyotrophic lateral sclerosis (ALS) in a subject, said method comprising administering a therapeutically effective amount of a composition comprising extracellular vesicles (EVs) derived from neural cells to the subject, thereby treating ALS.

2. The method of claim 1, wherein the composition is administered to a site distal to the central nervous system (CNS) of the subject.

3. The method of claim 2, wherein the composition is administered intravenously, intranasally, intraperitoneally, orally, or by combination of any thereof.

4. The method of any one of claims 1-3, wherein neurological performance, motor performance, and/or one or more signs or symptoms associated with ALS are improved, and/or survival is prolonged in the subject relative to a control subject.

5. The method of claim 4, wherein the one or more signs or symptoms comprise muscle weakness, walking difficulty, tripping, falling, hand weakness, hand clumsiness, slurred speech, chewing difficulty, swallowing difficulty, and/or breathing difficulty.

6. The method of any one of claims 1-5, wherein level or activity of neurofilament light chain, chitinase-3-like protein 1(CHI3L1 ), nucleotide-binding domain leucine-rich repeat containing family pyrin domain containing 3 (NLRP3), a NLRP3 signaling pathway molecule, and/or a proinflammatory cytokine is decreased in the subject.

7. The method of claim 6, wherein the NLRP3 signaling pathway molecule comprises caspase-1, IL1-β, and/or IL-18, and/or the proinflammatory cytokine comprises TNFα and/or IL1-β.

8. A method of inhibiting necroptosis in a cell, said method comprising contacting the cell with a therapeutically effective amount of a composition comprising extracellular vesicles (EVs) derived from neural cells, thereby inhibiting necroptosis.

9. The method of claim 8, wherein level of phosphorylated receptor interacting protein kinase 1 (p-RIPK1) is decreased in the cell relative to a control cell.

10. The method of claims 8 or 9, wherein the cell is within a subject.

11. The method of claim 10, wherein the subject has amyotrophic lateral sclerosis (ALS), and wherein neurological performance, motor performance, and/or one or more signs or symptoms associated with ALS are improved, and/or survival is prolonged in the subject relative to a control subject.

12. The method of any one of claims 1-11, wherein the neural cells are non-transformed neural progenitor cells.

13. The method of any one of claims 1-12, wherein the neural cells are derived from human pluripotent stem cells, human induced pluripotent stem cells, or human embryonic stem cells.

14. The method of any one of claims 1-13, wherein the EVs comprise ecto-5′-nucleotidase (CD73).

15. The method of claim 14, wherein the EVs further comprise one or more of melanoma-associated chondroitin sulfate proteoglycan (MCSP), pentraxin-3 (PTX3), angiopoietin-1, insulin-like growth factor-binding protein 2(IGFBP 2 ), and macrophage colony-stimulating factor.

16. The method of any one of claims 1-15, wherein the composition comprises about 1 mg to about 750 mg of EVs.

17. The method of any one of claims 1-16, wherein the EVs comprise exosomes.

18. The method of any one of claims 1-17, wherein the EVs are about 10 nm to about 10 μmin size.

19. The method of claim 18, wherein the EVs are about 20 nm to about 250 nm in size.

20. The method of any one of claims 1-19, wherein the EVs further comprise cargo.

21. The method of claim 20, wherein the cargo comprises one or more of a nucleotide, a peptide, a protein, an antibody, and a small molecule.

22. The method of claim 21, wherein the nucleotide is one or more of a siRNA, an antisense oligonucleotide, an mRNA, a plasmid, and a cDNA.
